(19) United States
(12) Patent Application Publication
(10) Pub. No.: US 2009/0215640 A1
(43) Pub. Date: Aug. 27, 2009

(54) METHODS FOR IDENTIFYING LIGANDS FOR STEM CELLS AND CELLS DERIVED THEREFROM

(76) Inventors: Michael D. West, Mill Valley, CA (US); Karen B. Chapman, Mill Valley, CA (US); David Larocca, Encinitas, CA (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 11/915,696

(22) PCT Filed: May 26, 2006
(86) PCT No.: PCT/US06/20552

§ 371 Date: Aug. 27, 2008

Related U.S. Application Data
(60) Provisional application No. 60/685,758, filed on May 27, 2005.

(51) Int. Cl. C40B 30/04; C12N 5/06

(52) U.S. Cl. 506/9; 435/325

(57) ABSTRACT

The present invention provides methods for the identification of novel ligands to pluripotent stem cells such as human embryonic stem cells, human embryo-derived cells, and from cells differentiated from such cells, and the use of such ligands in identifying differentiation conditions, purifying cells, and for eliminating such cells from mixtures of varied cell types. The invention also provides methods for the identification of target progenitor cells and cells identified thereby.

### CROSS-REFERENCE TO RELATED APPLICATIONS

This application claims the benefit of U.S. Provisional Application No. 60/685,758, filed on May 27, 2005, the disclosure of which is incorporated herein by reference in its entirety.

### BACKGROUND OF THE INVENTION

Advances in pluripotent stem cell technology, such as the use of human embryonic stem (hES) cells have become an important new focus of medical research. hES cells have a demonstrated potential to differentiate into any and all of the cell types in the human body including complex tissues. Other pluripotent stem cells, such as those cells downstream of ES cells that are of the endodermal, mesodermal, or ectodermal lineages will also likely be cultured to yield therapeutically-useful cells. This has led to the suggestion that diseases due to the dysfunction of cells may be amenable to treatment using cells derived from pluripotent stem cells.

While techniques to differentiate hES cells into numerous differentiated states have been described, there remains a need for methods to identify the specific cell types derived from pluripotent stem cells, including purified pluripotent stem cells that have already committed to specific endodermal, mesodermal, or ectodermal lineages.

Moreover, cell-cell and cell-matrix contacts can be important to proliferation and viability of ES cells, and in certain instances, to differentiating ES cells into various cell lineages of the body. However, little is known about which cellular factors are responsible for differentiation of ES cells into each lineage pathway. Such knowledge is essential for replicating differentiation in culture such that specific cells can be differentiated in sufficient quantities for cell therapy.

Because cells communicate with other cells and their immediate microenvironment through cell surface receptors, certain changes in the repertoire of cell surface receptors are likely to be specific for particular lineages. It would be of great interest to take advantage of these proteins to identify cells as they proliferate, differentiate, and/or undergo apoptosis. For example, surface markers are effective for identifying progenitors and precursors in the neural and hematopoietic compartments. For the latter, many well-characterized immunological reagents that recognize surface epitopes are routinely used to isolate self-renewing hematopoietic stem cells and multipotent and committed progenitor cells. See, e.g., Civin and Gore, J. Hematother. 2:137-144, 1993. Epidermal differentiation has also been studied using embryonic stem cell models, and the role of various growth factors and receptors in the process have been examined. See, e.g., Turken and Troy, Biochem. Cell Biol. 76:889-898, 1998. Understanding and characterizing the function of surface epitopes during the proliferation, differentiation, and/or apoptosis of these and other cells and tissues is clearly an important problem that remains to be fully understood. There also remains a need for novel methods to identify ligands that bind to cell surface receptors during these processes.

### SUMMARY OF THE INVENTION

The present invention is directed to the use of display libraries to identify ligands, preferably peptide or polypeptide ligands, that bind selective populations of progenitor cells. The invention is also directed to the use of display libraries to identify target progenitor cells. The invention is also directed to ligands and progenitor cells identified by the above methods. The ligands of the invention may be selective for cells that are at particular stages of proliferation, differentiation, and/or apoptosis, or that are predisposed to differentiate along particular paths. The progenitor cells of the invention may be from stem cells, such as embryonic stem cells or other embryo-derived cells such as inner cell mass-derived cells, or adult-derived stem cells. The progenitor cells may be totipotent, or may be pluripotent though having already committed to endodermal, mesodermal, or ectodermal lineages. The progenitor cells may also be generated by de-differentiation of differentiated cells.

Thus, in one aspect, the invention provides a method for identifying a ligand that binds a target progenitor cell, comprising the steps of:
(i) providing a ligand display library comprising a plurality of display packages, each display package comprising at least one test ligand disposed on the surface of the display package;
(ii) contacting the display library with the target progenitor cell;
(iii) allowing the progenitor cell to differentiate; and
(iv) identifying the at least one test ligand disposed on the surface of a display package associated with a differentiated cell.

In certain embodiments, the method further comprises the step of isolating a differentiated cell with an associated display package prior to identifying the at least one test ligand.

In certain specific embodiments, the associated display package is bound to the surface of the differentiated cell. In other specific embodiments, the associated display package has been internalized into the differentiated cell by receptor-mediated endocytosis.

In some embodiments, differentiation of the progenitor cell is induced prior to identifying the at least one test ligand. In specific embodiments, the at least one test ligand disposed on the surface of the associated display package selectively induces differentiation of the progenitor cell. In some embodiments, differentiation of the progenitor cell is inhibited prior to identifying the at least one test ligand. In specific embodiments, the at least one test ligand disposed on the surface of the associated display package selectively inhibits differentiation of the progenitor cell.

In some embodiments, proliferation of the progenitor cell is induced prior to identifying the at least one test ligand. In specific embodiments, the at least one test ligand disposed on the surface of the associated display package selectively induces proliferation of the progenitor cell. In some embodiments, proliferation of the progenitor cell is inhibited prior to identifying the at least one test ligand. In specific embodiments, the at least one test ligand disposed on the surface of the associated display package selectively inhibits proliferation of the progenitor cell.

In some embodiments, apoptosis of the progenitor cell is induced prior to identifying the at least one test ligand. In specific embodiments, the at least one test ligand disposed on the surface of the associated display package selectively induces apoptosis of the progenitor cell. In some embodiments, apoptosis of the progenitor cell is inhibited prior to identifying the at least one test ligand. In specific embodiments, the at least one test ligand disposed on the surface of the associated display package selectively inhibits apoptosis of the progenitor cell.

In some embodiments of the invention, the display package comprises no more than 5-10%, no more than 2%, or no more than 1% polyvalent displays. In some embodiments, the at least one test ligand disposed on the surface of the display package is a peptide ligand. In specific embodiments, the peptide ligand is 4-20 amino acid residues in length.

In some embodiments of the invention, the plurality of display packages is a plurality of phage particles. In more specific embodiments, the phage particles are selected from the group consisting of M13, f1, fd, If1, Ike, Xf, Pf1, Pf3, λ, T4, T7, P2, P4, ΦX174, MS2 and f2. In yet more specific embodiments, the phage particles are filamentous bacteriophage specific for Escherichia coli and comprise a phage coat protein selected from the group consisting of coat proteins III, VI, VII, VIII, and IX. In even more specific embodiments, the filamentous bacteriophage is selected from the group consisting of M13, fd, and f1.

In other embodiments of the invention, the plurality of display packages is a plurality of bacteria or a plurality of spores.

In some embodiments of the invention, the ligand display library comprises at least 10, at least 100, at least 1000, or at least 10,000 different display packages, each display package comprising at least one test ligand disposed on the surface of the display package.

In some embodiments, the display package associated with the differentiated cell is identified at least 1 day, at least 2 days, at least 4 days, at least 6 days, at least 12 days, or at least 18 days after contacting the display packages with the target progenitor cell.

In some embodiments, at least one of the display packages comprises a plurality of test ligands disposed on the surface of the display package.

In some embodiments, the identifying step comprises amplification. In specific embodiments, the amplification is by replication. In other specific embodiments, the amplification is by nucleic acid amplification.

In some embodiments of the invention, the target progenitor cell is a human embryo-derived cell. In other embodiments, the target progenitor cell is a human ES cell. In some embodiments, the target progenitor cell is a canine or feline target progenitor cell. In some embodiments, the target progenitor cell is provided in a culture of stem cells or cultured embryos, or explanted tissues that contain stem cells. In some embodiments, the target progenitor cell is a mesodermal pluripotent stem cell, an ectodermal pluripotent stem cell, or an endodermal pluripotent stem cell. In some embodiments, the target progenitor cell is a dermal cell with a prenatal pattern of gene expression. In other embodiments, the target progenitor cell is a hematopoietic stem cell with a prenatal pattern of gene expression. In still other embodiments, the target progenitor cell is a progenitor of a retinal pigment epithelial cell.

In another aspect, the invention provides a ligand identified by the above methods.

In another aspect, the invention provides a target progenitor cell that selectively binds a ligand identified by the above methods.

In yet another aspect, the invention provides a method for identifying a target progenitor cell, comprising the steps of:
(i) providing a ligand display library comprising a plurality of display packages, each display package comprising at least one test ligand disposed on the surface of the display package;
(ii) contacting the display library with a target progenitor cell;
(iii) allowing the progenitor cell to differentiate; and
(iv) identifying a differentiated cell that associates a display package.

In certain embodiments, the method further comprises the step of identifying the at least one test ligand disposed on the surface of the associated display package.

In another aspect, the invention provides a target progenitor cell identified by the above methods.

### LISTING OF DRAWINGS

FIG. 1. Enrichment of mESC-binding phage particles.

FIG. 2. Schematic depiction of time-lapse phage display. Cell surface markers of various progenitors are identified by adding a phage display library to differentiating cells at regular intervals during the differentiation of ESCs and recovering the cells at a later time when differentiated cells are present. The result is a time lapse map of the appearance of various markers on progenitor cells as they occur over time.

FIG. 3. Time-lapse selection strategy.

FIG. 4. Recovery of internalizing peptide phage particles after prolonged incubation of target cells.

FIG. 5. Tracking of peptide-targeted embryonic cells using Q-dot labeled peptide phage.

FIG. 6. Regulation of PCSK1N, PCSK5, and PCSK9 expression in clonal hESC-derived lines.

### DETAILED DESCRIPTION OF THE INVENTION

Phage display is a powerful technology that has been used successfully to identify cell-binding ligands and their receptors. Brown, Curr. Opin. Chem. Biol. 4(1):16-21, 2000; Larocca and Baird, Drug Discov. Today 6(15):793-801, 2001. Phage display libraries have been used to identify peptides with high selectivity for endothelial populations in various organs and tumors. Ruoslahti and Rajotte, Annu. Rev. Immunol. 18:813-827, 2000. Phage display libraries have been used in mouse to identify a peptide that homes to bone marrow and that binds to hematopoietic stem cells. Nowakowski et al., Stem Cells 22:1030-1038, 2004. Selection from phage libraries is thus a useful complement to genomic approaches, which have inherent limitations, particularly when used to characterize heterogeneous cell populations. See, e.g., King and Sinha, JAMA 286:2280-2288, 2001. The selected phage particles as well as their encoded cell-binding peptide ligands provide useful affinity reagents for cell detection and purification. Other examples of the usefulness of phage display for various purposes are described in U.S. Pat. Nos. 6,448,083; 6,450,527; 6,472,146; 6,589,730; 6,723,512; and U.S. Patent Application Publication No. 2003/0148263.

Display technologies arose nearly 20 years ago from the observation that filamentous bacteriophage can be genetically modified to display a variety of peptide or protein ligands as fusions to phage coat proteins. Smith, Science 228(4705):1315-1317, 1985. A key advantage of display on a genetic particle is that it links the phenotype of the displayed protein with its genotype encoded in the phage genome. The simple genome and rapid replication cycle of bacteriophage allows for the construction of very large combinatorial display libraries typically consisting of hundreds of millions of highly diverse peptides or proteins from which binding ligands can be selected. Over the past 15 years, selection from phage display libraries has proven to be a valuable method of selecting binding ligands against simple and complex targets. Brown, Curr. Opin. Chem. Biol. 4(1):16-21, 2000; Smith and Petrenko, Chemical Reviews 97(s):391-410, 1985.

The typical general strategy for identifying ligands from phage display libraries is to perform an affinity selection to purify those phage particles that most tightly bind to a given target. Following incubation of the phage library with the target, non-binders are removed through repeated washing. The binding phage particles are then released from the target by washing, for example, with low pH buffer or chaotropic agents. The recovered phage particles are amplified by infection and subsequent replication in a suitable bacterial host. The amino acid sequences of putative binding ligands are obtained by sequencing DNA from a random sample of recovered phage clones at each round of selection. The process is repeated until the complexity of the library is sufficiently reduced such that individual binding phage clones can be identified and further characterized.

Selection of high affinity ligands against purified molecular targets from phage display libraries has been widely successful. Smith and Petrenko, Chemical Reviews 97(s):391-410, 1985. There are many examples of successful selection of peptides that target various cell types including adult cardiomyocytes using a variety of selection strategies. Nicklin et al, Circulation 102(2):231-237, 2000; Oyama et al., Cancer Lett. 202(2):219-230, 2003; McGuire et al., J. Mol. Biol. 342(1):171-182, 2004; Romanov et al., Prostate 47(4):239-251, 2001. The technique of in-vivo phage selection has been used to identify vascular addresses within whole organs. Rajotte et al., J. Clin. Invest. 102(2):430-437; Essler and Ruoslahti, Proc. Natl. Acad. Sci. USA 99(4):2252-2257, 2002; Zhang et al., Circulation 112:1601-1611, 2005. A key advantage of selection against cells or organs is that it does not require prior knowledge of the targeted receptor.

Improved strategies for selection on cells have been developed that rely on ligand internalization (Barry et al., Nat. Med. 2:299-305, 1996; Poul et al., J. Mol. Biol. 301(5):1149-1161, 2000) and phage-mediated gene delivery (Kassner et al., Biochem. Biophys. Res. Commun. 64(3):921-928, 1999; Legendre and Fastrez, Gene 290(1-2):203-215, 2002). Internalization allows for a more stringent selection that reduces the problem of high background due to non-specific binding phage particles since these phage particles can be removed from the surface with stringent washing after internalization has occurred. However, several studies indicate that internalized phage particles may rapidly lose infectivity because of proteolysis that occurs during both in-vitro and in-vivo selection. Barry et al., Nat. Med. 2:299-305, 1996; Molenaar et al., Virology 293(1):182-191, 2002. Even without internalization, phage particles are subject to digestion by extracellular proteases and the possible detrimental effects on infectivity. By far, the most common method of recovering and amplifying selected phage particles during display library selection is infection of host bacteria, as described by Smith 20 years ago. Smith, Science 228(4705):1315-1317, 1985. While this method of recovery is suitable for most phage selections, it may not be optimal when selecting phage libraries on live target cells or in other approaches where phage viability may be compromised.

To overcome problems associated with loss of phage infectivity and to increase selection sensitivity, Burg et al. developed an alternative strategy that employs φ29 rolling circle amplification (RCA) of circular phage DNA for the recovery of ligand display phage. Burg et al., DNA and Cell Biology, 23(7):457-462, 2004. Unlike recovery by infectivity, DNA amplification recovers all internalized phage particles regardless of infectivity by extracting the phage DNA from treated cells and preferentially amplifying the circular phage DNA with φ29 DNA polymerase mediated RCA. Dean et al., Genome Res. 11(6):1095-1096, 2001. PCR may also be effectively used for phage recovery (Kassner et al., Biochem. Biophys. Res. Commun. 64(3):921-928, 1999) and is better suited for amplification of linear T7 phage DNA. Burg et al. have compared recovery by DNA amplification directly to standard recovery by infectivity in a model system and find it more sensitive at selecting cell-targeting ligands. Burg et al., DNA and Cell Biology, 23(7):457-462, 2004. Thus efficient recovery of selected phage particles by DNA amplification has been successfully demonstrated.

The present invention makes available a powerful directed approach for isolating ligands, in certain embodiments peptide ligands, that bind selective populations of progenitor cells, and that may be selective for cells at particular stages of proliferation, differentiation, and/or apoptosis, or for cells that are predisposed to differentiate along particular paths.

Utilizing display techniques, a ligand library may first be reduced in complexity by panning or other affinity purification techniques. In particular, the subject method selects ligand having a certain affinity profile, e.g., a specificity and/or binding affinity for a discrete target progenitor cell by (i) displaying the ligands on the outer surface of an identifiable display package, in certain embodiments a replicable genetic display package, to create a ligand display library, and (ii) using affinity and/or functional activity selection techniques to enrich the population of display packages for those containing ligands that have a desired binding specificity for and/or biological effect on the target cell.

Before further description of the invention, certain terms employed in the specification, examples and appended claims are, for convenience, collected here.

The term “ligand” refers to a chemical entity that interacts specifically or selectively with at least one receptor on the surface of or within at least one target progenitor cell.

The term “peptide” refers to an oligomer in which the monomers are amino acids (usually alpha-amino acids) joined together through amide bonds. Peptides are two or more amino acid monomers long, but more often are between 5 to 10 amino acid monomers long and can be even longer, i.e. up to 20 amino acids or more, although peptides longer than 20 amino acids are more likely to be called “polypeptides.” The term “protein” is well known in the art and usually refers to a very large polypeptide, or set of associated homologous or heterologous polypeptides, that has some biological function. However, for purposes of the present invention the terms “peptide,” “polypeptide,” and “protein” are largely interchangeable as all three types can be used to generate the display library and so are collectively referred to as “peptides”.

The term “random peptide library” refers to a set of random or semi-random peptides, as well as sets of fusion proteins containing those random peptides (as applicable).

The term “ligand display package” refers to a particle that contains at least one ligand disposed on its surface in such a way that the ligand can interact with a receptor on the surface of or within a target progenitor cell. The ligand display package is in certain embodiments identifiable, so that the specific ligand or ligands disposed on any particular particle may be identified. In certain specific embodiments, the ligand display package is a “peptide display package”.

The language “replicable genetic display package” describes one example of a ligand display package. A replicable genetic display package is a biological particle that has genetic information providing the particle with the ability to replicate. The package may, for example, display a fusion protein including a peptide derived from the variegated peptide library. The test peptide portion of the fusion protein is presented by the display package in a context that permits the peptide to bind to a target that is contacted with the display package. The display package will generally be derived from a system that allows the sampling of very large variegated peptide libraries. The display package may be, for example, derived from vegetative bacterial cells, bacterial spores, or bacterial viruses.

As used herein, “variegated” refers to the fact that a population of ligands is characterized by having a ligand structure which differs from one member of the library to the next. In certain embodiments of the present invention, the ligand display collectively produces a ligand library including at least 96 to 107 different ligands, so that diverse ligands may be simultaneously assayed for the ability to interact with the target progenitor cell.

The language “differential binding means”, as well as “affinity selection” and “affinity enrichment”, refer to the separation of members of the ligand display library based on the differing abilities of ligands on the surface of each of the display packages of the library to bind to the target cell. The differential binding of a target progenitor cell by test ligands of the display may be used in the affinity separation of those ligands that specifically bind the target cell from those that do not. For example, the affinity selection protocol may also include a pre- or post-enrichment step wherein display packages capable of binding “background targets”, e.g., as a negative selection, are removed from the library.

The term “solid support” refers to a material having a rigid or semi-rigid surface. Such materials will preferably take the form of small beads, pellets, disks, chips, dishes, multi-well plates, wafers or the like, although other forms may be used. In some embodiments, at least one surface of the substrate will be substantially flat. The term “surface” refers to any generally two-dimensional structure on a solid substrate and may have steps, ridges, kinks, terraces, and the like without ceasing to be a surface.

The language “fusion protein” and “chimeric protein” are art-recognized terms which are used interchangeably herein, and include contiguous polypeptides comprising a first polypeptide covalently linked via an amide bond to one or more amino acid sequences that define polypeptide domains that are foreign to and not substantially homologous with any domain of the first polypeptide. One portion of the fusion protein may comprise a test peptide, e.g., a peptide that has a random or semi-random sequence. A second polypeptide portion of the fusion protein may be derived from an outer surface protein or display anchor protein that associates the test peptide with the outer surface of the peptide display package. As described below, where the display package is a phage particle, this anchor protein may be derived from a surface protein native to the genetic package, such as a viral coat protein. Where the fusion protein comprises a viral coat protein and a test peptide, it will be referred to as a “peptide fusion coat protein”. The fusion protein may further comprise a signal sequence, which is a short length of amino acid sequence at the amino terminal end of the fusion protein that directs at least the portion of the fusion protein including the test peptide to be secreted from the cytosol of a cell and localized on the extracellular side of the cell membrane.

Gene constructs encoding fusion proteins are likewise referred to a “chimeric genes” or “fusion genes”.

The term “vector” refers to a DNA molecule, capable of replication in a host cell, into which a gene may be inserted to construct a recombinant DNA molecule.

The terms “phage vector” and “phagemid” are art-recognized and generally refer to a vector derived by modification of a phage genome, containing an origin of replication for a bacteriophage, and, in certain embodiments, an origin (ori) for a bacterial plasmid. The use of phage vectors rather than the phage genome itself, provides greater flexibility to vary the ratio of chimeric peptide/coat protein to wild-type coat protein, as well as supplement the phage genes with additional genes encoding other heterologous polypeptides, such as “auxiliary polypeptides” which may be useful in the “dual” peptide display constructs described below.

The language “helper phage” describes a phage particle that is used to infect cells containing a defective phage genome or phage vector and that functions to complement the defect. The defect can be one which results from removal or inactivation of phage genomic sequence required for production of phage particles. Examples of helper phage are M13K07.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences may be directly or indirectly regulated by the target receptor protein. Exemplary transcriptional control sequences are promoter sequences. A reporter gene is meant to include a promoter-reporter gene construct that is heterologously expressed in a cell.

The term “teratoma” refers to a benign mass of cells differentiating from pluripotent stem cells that organize into complex tissues in three dimensions, though lacking the normal and intact form of an animal and incapable of independent life. By way of example, teratomas have been reported to occur following the injection of hES cells into the skeletal muscle or peritoneum of immunocompromised mice where such teratomas contain intestine, skin, teeth, renal tissue, neuronal tissue, bone, cartilage, and so on. A teratoma, as referred to in this specification, may be the result of cells being cultured in vivo or in vitro.

The term “pluripotent stem cells” refers to animal cells capable of differentiating into more than one differentiated cell type. Such cells include hES cells, hEDCs, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification.

The term “embryonic stem cells” (ES cells or ESCs) refers to cells derived from the inner cell mass of blastocysts or morulae that have been serially passaged as cell lines. The ES cells may be derived from fertilization of an egg cell with sperm or DNA, nuclear transfer, parthenogenesis, or by means to generate ES cells with homozygosity in the MHC region. The term “human embryonic stem cells” (hES cells or hESCs) refers to human ES cells.

The term “embryo-derived cells” (ED cells or EDCs) refer to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives, but excluding ES cells that have been passaged as cell lines. The ED cells may be derived from fertilization of an egg cell with sperm or DNA, nuclear transfer, chromatin transfer, parthenogenesis, analytical reprogramming technology, or by means to generate ES cells with homozygosity in the HLA region. The term “human embryo-derived cells (hED cells or hEDCs) refers to human ED cells.

The term “embryonic germ cells” (EG cells or EGCs) refer to pluripotent stem cells derived from the primordial germ cells of fetal tissue, that can differentiate into various tissues in the body. The EG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. Patent Application Nos. 60/161,987, filed Oct. 28, 1999; Ser. Nos. 09/697,297, filed Oct. 27, 2000; 09/995,659, filed Nov. 29, 2001; 10/374,512, filed Feb. 27, 2003; PCT International Application No. PCT/US/00/29551, filed Oct. 27, 2000; the disclosures of which are incorporated herein in their entireties). The term “human embryonic germ cells” (hEG cells or hEGCs) refers to human EG cells.

The term “progenitor cell” refers to any cell that is capable of undergoing differentiation, including cells that undergo changes in proliferative capacity and/or apoptosis. It includes undifferentiated cells, such as, for example, embryonic stem cells, inner cell mass cells, embryo-derived cells, embryonal carcinoma cells, teratocarcinoma cells, blastomeres, and germ-line cells. The term also includes differentiated cells that have been, or are in the process of being, de-differentiated, for example by the methods disclosed in U.S. Patent Application Publication Nos. 2002/0001842; 2004/0199935; 2003/0044976; and PCT International Publication Nos. WO 01/00650; WO 03/018780; WO 2004/094611; and WO 2005/049788; all of which are incorporated by reference herein in their entireties.

The term “analytical reprogramming technology” refers to a variety of methods to reprogram the pattern of gene expression of a somatic cell to that of a more pluripotent state, such as that of an ES, ED, or EG cell, wherein the reprogramming occurs in multiple and discrete steps and does not rely simply on the transfer of a somatic cell into an oocyte and the activation of that oocyte (see U.S. application Nos. 60/332,510, filed Nov. 26, 2001; 10/304,020, filed Nov. 26, 2002; PCT application no. PCT/US02/37899, filed Nov. 26, 2003; U.S. application No. 60/705,625, filed Aug. 3, 2005, the disclosure of each of which is incorporated by reference in their entirety).

Throughout this specification and claims, the word “comprise”, or variations such as “comprises” or “comprising” will be understood to imply the inclusion of a stated integer or group of integers but not the exclusion of any other integer or group of integers.

### Generation of Ligand Libraries

The ligand libraries of the instant invention are disposed on the surface of display packages such that the ligands may interact with at least one receptor on at least one target progenitor cell. The ligands are preferably disposed on the surface of the display packages by covalent attachment, although other forms of display are also within the scope of the invention. The ligands may be synthesized, for example by combinatorial chemical synthesis, prior to their disposal on the surface of a display package, or they may synthesized directly on the surface of the display package itself, for example by stepwise chemical coupling, as is well-known by those of skill in the art of nucleic acid and peptide synthesis. The ligands may alternatively or in combination be synthesized by biological methods, for example by expression on the surface of a biological display package, such as, for example, a phage particle or bacterial cell.

In a preferred embodiment of the invention, the ligand display libraries are variegated peptide libraries expressed on the surface of a biological display package. The variegated peptide libraries may be generated by any of a number of methods, including those exploiting recent trends in the preparation of chemical libraries. See below.

In one embodiment, a test peptide library is generated to express a combinatorial library of peptides that is not based on any known sequences, nor derived from cDNA. That is, the sequences of the library are largely, if not entirely, random. It will be evident that the peptides of the library may range in size from dipeptides to large proteins.

In another embodiment, the peptide library is generated to express a combinatorial library of peptides that is based at least in part on one or more known polypeptide sequences or portions thereof. That is, the sequences of the library are semi-random, being derived by combinatorial mutagenesis of a known sequence(s). See, for example, Ladner et al. PCT International Publication No. WO 90/02909; Garrard et al., International Publication No. WO 92/09690; Marks et al., J. Biol. Chem. 267:16007-16010, 1992; Griffths et al., EMBO J. 12:725-734, 1993; Clackson et al., Nature 352:624-628, 1991; and Barbas et al., Proc. Natl. Acad. Sci. USA 89:4457-4461, 1992. Accordingly, polypeptide(s) that are known ligands for a target protein may be mutagenized by standard techniques to derive a variegated library of polypeptide sequences that may further be screened for binding activity. The purpose of screening such combinatorial peptide libraries is to generate, for example, homologs of known polypeptides that may act as ligands for the target protein, or alternatively, possess novel activities all together. To illustrate, a ligand may be engineered by the present method to provide more efficient binding or specificity to a cognate receptor, yet still retain at least a portion of an activity associated with the wild-type ligand. Thus, combinatorially-derived homologs may be generated to have an increased potency relative to a naturally occurring form of the protein. Likewise, homologs may be generated by the present approach to act as antagonists, in that they are able to mimic, for example, binding to the target, yet not induce any biological response, thereby inhibiting the action of authentic ligand.

In certain preferred embodiments, the combinatorial polypeptides are in the range of 3-100 amino acids in length, more preferably at least 5-50, and even more preferably at least 7, 10, 13, 15, 20 or 25 amino acid residues in length. Preferably, the polypeptides of the library are of uniform length. It will be understood that the length of the combinatorial peptide does not reflect any extraneous sequences that may be present in order to facilitate expression, e.g. such as signal sequences or invariant portions of a fusion protein.

The harnessing of biological systems for the generation of peptide diversity is now a well established technique that may be exploited to generate the peptide libraries of the subject method. The source of diversity is the combinatorial chemical synthesis of mixtures of oligonucleotides. Oligonucleotide synthesis is a well-characterized chemistry that allows tight control of the composition of the mixtures created. Degenerate DNA sequences produced are subsequently placed into an appropriate genetic context for expression as peptides.

There are at least two principal ways in which to prepare the required degenerate mixture. In one method, the DNAs are synthesized a base at a time. When variation is desired at a base position dictated by the genetic code a suitable mixture of nucleotides is reacted with the nascent DNA, rather than the pure nucleotide reagent of conventional polynucleotide synthesis. The second method provides more exact control over the amino acid variation. First, trinucleotide reagents are prepared, each trinucleotide being a codon of one (and only one) of the amino acids to be featured in the peptide library. When a particular variable residue is to be synthesized, a mixture is made of the appropriate trinucleotides and reacted with the nascent DNA. Once the necessary “degenerate” DNA is complete, it is joined with the DNA sequences necessary to assure the expression of the peptide, as discussed in more detail below, and the complete DNA construct is introduced into the cell.

Whatever the method used for generating diversity at the codon level, chemical synthesis of a degenerate gene sequence may be carried out in an automatic DNA synthesizer, and the synthetic genes may then be ligated into an appropriate gene for expression. The purpose of a degenerate set of genes is to provide, in one mixture, all of the sequences encoding the desired set of potential test peptide sequences. The synthesis of degenerate oligonucleotides is well known in the art (see for example, Narang, Tetrahedron 39:3, 1983; Itakura et al., Recombinant DNA, Proc 3rd Cleveland Sympos. Macromolecules, ed. AG Walton, Amsterdam: Elsevier, pp. 273-289, 1981; Itakura et al, Annu. Rev. Biochem. 53:323, 1984; Itakura et al., Science 198:1056, 1984; Ike et al., Nucleic Acids Res. 11:477, 1983. Such techniques have been employed in the directed evolution of other proteins (see, for example, Scott et al., Science 249:386-390, 1990; Roberts et al., Proc. Natl. Acad. Sci. USA 89:2429-2433, 1992; Devlin et al., Science 249: 404-406, 1990; Cwirla et al., Proc. Natl. Acad. Sci. USA 87: 6378-6382, 1990; as well as U.S. Pat. Nos. 5,223,409; 5,198,346; and 5,096,815).

### Ligand Display Formats

As previously mentioned, the ligand libraries of the instant invention are disposed on the surface of a display package. The display package provides a surface on which the ligands are presented to the target progenitor cell. The display package is preferably identifiable, so that the ligand or ligands disposed on the surface of the display package may be identified after the display package has contacted the target progenitor cell and the progenitor cell has proliferated, differentiated, and/or undergone apoptosis.

In some embodiments, the display package is, for example, a bead or microsphere with at least one test ligand disposed on its surface. In these embodiments, the bead or microsphere includes a tag that allows the bead or microsphere, and the ligand disposed thereon, to be uniquely identified. In some embodiments, the tag may comprise an optically interrogatable encoding scheme. See, e.g., U.S. Pat. Nos. 6,023,540 and 6,327,410. In other embodiments, structural information about the test ligand may be obtained by mass spectrometric analysis. See, e.g., U.S. Pat. Nos. 6,475,807, 6,625,546. Other methods of identifying a specific test ligand from a library of test ligands are known to those of skill in the art. See, e.g., Janda, Proc. Natl. Acad. Sci. USA 91:10779-10785, 1994.

In a preferred embodiment, the ligand display library is an expressed peptide display library. With respect to the display package on which the variegated peptide library is manifest, it will be appreciated from the discussion provided herein that the display package will preferably be able to be (i) genetically altered to encode a variegated peptide library, (ii) maintained and amplified in culture, (iii) manipulated to display the variegated peptide library on the surface of the display package in a manner permitting the displayed peptides to interact with a target during an affinity separation step, and (iv) affinity separated while retaining the nucleotide sequence encoding the displayed peptide such that the sequence of the peptide gene may be obtained. In preferred embodiments, the display remains viable after affinity separation.

Ideally, the display package comprises a system that allows the sampling of very large variegated peptide display libraries, rapid sorting after each affinity separation round, and easy isolation of the peptide gene from purified display packages or further manipulation of that sequence in a secretion mode (described below). The most attractive candidates for this type of screening are prokaryotic organisms and viruses, as they can be amplified quickly, they are relatively easy to manipulate, and large number of clones may be created. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages.

In addition to commercially available kits for generating phage display libraries (e.g. the Pharmacia Recombinant Phage Antibody System, catalog no. 27-9400-01; and the Stratagene SurfZAP™ phage display kit, catalog no. 240612), examples of methods and reagents particularly amenable for use in generating the variegated peptide display library of the present invention may be found in, for example, the Ladner et al. U.S. Pat. No. 5,223,409; the Kang et al. International Publication No. WO 92/18619; the Dower et al. International Publication No. WO 91/17271; the Winter et al. International Publication No. WO 92/20791; the Markland et al International Publication No. WO 92/15679; the Breitling et al. International Publication No. WO 93/01288; the McCafferty et al. International Publication No. WO 92/01047; the Garrard et al. International Publication No. WO 92/09690; the Ladner et al. International Publication No. WO 90/02809; Fuchs et al., Bio/Technology 9:1370-1372, 1991; Hay et al., Hum. Antibod. Hybridomas 3:81-85, 1992; Huse et al., Science 246:1275-1281, 1989; Griffths et al., EMBO J. 12:725-734, 1993; Hawkins et al., J. Mol. Biol. 226:889-896, 1992; Clackson et al, Nature 352:624-628, 1991; Gram et al., Proc. Natl. Acad. Sci. USA 89:3576-3580, 1992; Garrad et al., Bio/Technology 9:1373-1377, 1991; Hoogenboom et al., Nuc. Acids Res. 19:4133-4137, 1991; and Barbas et al., Proc. Natl. Acad. Sci. USA 88:7978-7982, 1991. These systems may, with modifications described herein, be adapted for use in the subject method.

When the display is based on a bacterial cell, or a phage particle that is assembled periplasmically, the display means of the package will comprise at least two components. The first component is a secretion signal that directs the recombinant peptide to be localized on the extracellular side of the cell membrane (of the host cell when the display package is a phage particle). This secretion signal may be selected so as to be cleaved off by a signal peptidase to yield a processed, “mature” peptide. The second component is a display anchor protein that directs the display package to associate the test peptide with its outer surface. As described below, this anchor protein may be derived from a surface or coat protein native to the genetic package.

When the display package is a bacterial spore, or a phage particle whose protein coating is assembled intracellularly, a secretion signal directing the peptide to the inner membrane of the host cell may be unnecessary. In these cases, the means for arraying the variegated peptide library comprises a derivative of a spore or phage coat protein amenable for use as a fusion protein.

In some instances it may be necessary to introduce an unstructured polypeptide linker region between portions of the chimeric protein, e.g., between the test peptide and display polypeptide. This linker may facilitate enhanced flexibility of the chimeric protein allowing the test peptide to freely interact with a target by reducing steric hindrance between the two fragments, as well as allowing appropriate folding of each portion to occur. The linker may be of natural origin, such as a sequence determined to exist in random coil between two domains of a protein. Alternatively, the linker may be of synthetic origin. For instance, the sequence (Gly₄Ser)₃ may be used as a synthetic unstructured linker. Linkers of this type are described in Huston et al., Proc. Natl. Acad. Sci. USA 85:4879, 1988; and U.S. Pat. Nos. 5,091,513 and 5,258,498. Naturally occurring unstructured linkers of human origin are preferred as they reduce the risk of immunogenicity.

In the instance wherein the display package is a phage, the cloning site for the test peptide gene sequences in the phagemid should be placed so that it does not substantially interfere with normal phage function. One such locus is the intergenic region as described by Zinder and Boeke, Gene 19:1-10, 1982.

The number of possible combinations in a peptide library may become large as the length is increased and selection criteria for degenerating at each position is relaxed. The ability to sample as many combinations as possible therefore depends, in part, on the ability to recover large numbers of transformants. For phage with plasmid-like forms (as filamentous phage), electrotransformation provides an efficiency comparable to that of phage-transfection with in vitro packaging, in addition to a very high capacity for DNA input. This allows large amounts of vector DNA to be used to obtain very large numbers of transformants. The method described by Dower et al., Nucleic Acids Res. 16:6127-6145, 1988, for example, may be used to transform fd-tet derived recombinants at the rate of about 10⁷ transformiants/ug of ligated vector into E. coli (such as strain MC1061), and libraries may be constructed in fd-tet Bl of up to about 3×10⁸ members or more. Increasing DNA input and making modifications to the cloning protocol within the ability of the skilled artisan may produce increases of greater than about 10-fold in the recovery of transformants, providing libraries of up to 10¹⁰ or more recombinants.

As will be apparent to those skilled in the art, in embodiments wherein high affinity ligands are sought, an important criteria for the present selection method may be that it is able to discriminate between ligands of different affinity for a particular target progenitor cell and preferentially enrich for the ligands of highest affinity. Applying the well known principles of ligand affinity and valence (i.e. avidity), it is understood that manipulating the display package to be rendered effectively monovalent may allow affinity enrichment to be carried out for generally higher binding affinities (i.e. binding constants in the range of 10⁶ to 10¹⁰ M⁻¹) as compared to the broader range of affinities isolable using a multivalent display package. To generate a monovalent display, the test ligands are disposed on the surface of the display packages at densities of, on average, approximately one test ligand per package. For example, in the case of phage libraries, the natural (i.e. wild-type) form of the surface or coat protein used to anchor the peptide to the display may be added at a high enough level that it almost entirely eliminates inclusion of the peptide fusion protein in the display package. Thus, a vast majority of the display packages may be generated to include no more than one copy of the peptide fusion protein (see, for example, Garrad et al. (1991) Bio/Technology 9:1373-1377). The density of ligands on other types of display packages may be adjusted to the same effect by modifying chemical coupling conditions or other parameters during the generation of the ligand display library.

In a preferred embodiment of a monovalent display library, the library of display packages will comprise no more than 5 to 10% polyvalent displays, and more preferably no more than 2% of the display will be polyvalent, and most preferably, no more than 1% polyvalent display packages in the population. In the case of phage libraries, the source of the wild-type anchor protein may be, for example, provided by a copy of the wild-type gene present on the same construct as the peptide fusion protein, or provided by a separate construct altogether. However, it will be equally clear that by similar manipulation, polyvalent displays may be generated to isolate a broader range of binding affinities. Such ligands may be useful, for example, in purification protocols where variation in avidity may be desirable.

While monovalent display may be preferred for selecting high affinity ligands, it may not always be preferred when selecting on cells. In some embodiments of the invention, selection by internalization may be used to identify cell-specific ligands. In many cases, multivalent display packages internalize more efficiently because they dimerize or multimerize the receptor which can trigger internalization (Becerril et al., Biochem. Biophys. Res. Commun. 255: 386-393, 1999; Ivanenkov et al., Biochimica et Biophysica Acta 1448: 463-472, 1999; Larocca et al., Molecular Therapy 3(4): 476-484, 2001; and Poul et al., J. Mol. Biol. 301(5):1149-61, 2000).

### Phage as Display Packages

Bacteriophage are attractive prokaryotic-related organisms for use in the subject method. Bacteriophage are excellent candidates for providing a display system of the variegated peptide library as there is little or no enzymatic activity associated with intact mature phage, and because their genes are inactive outside a bacterial host, rendering the mature phage particles metabolically inert. In general, the phage surface is a relatively simple structure. Phage can be grown easily in large numbers, they are amenable to the practical handling involved in many potential mass screening programs, and they carry genetic information for their own synthesis within a small, simple package. As the peptide gene is inserted into the phage genome, choosing the appropriate phage to be employed in the subject method will generally depend most on whether (i) the genome of the phage allows introduction of the peptide gene either by tolerating additional genetic material or by having replaceable genetic material; (ii) the virion is capable of packaging the genome after accepting the insertion or substitution of genetic material; and (iii) the display of the peptide on the phage surface does not disrupt virion structure sufficiently to interfere with phage propagation.

A further concern presented with the use of phage is that the morphogenetic pathway of the phage determines the environment in which the peptide will have opportunity to fold. Periplasmically assembled phage are preferred as the displayed peptides may contain essential disulfides, and such peptides may not fold correctly within a cell. However, in certain embodiments in which the display package forms intracellularly (e.g., where λ phage are used), it has been demonstrated in other instances that disulfide-containing peptides may assume proper folding after the phage is released from the cell.

Yet another concern related to the use of phage, but also pertinent to the use of bacterial cells and spores as well, is that multiple infections could generate hybrid displays that carry the gene for one particular test peptide yet have two or more different test peptides on their surfaces. Therefore, it may be preferable, though optional, to minimize this possibility by infecting cells with phage under conditions resulting in a low level of multiple infection.

For a given bacteriophage, the preferred display means is a protein that is present on the phage surface (e.g. a coat protein). Filamentous phage may be described by a helical lattice; isometric phage, by an icosahedral lattice. Each monomer of each major coat protein sits on a lattice point and makes defined interactions with each of its neighbors. Proteins that fit into the lattice by making some, but not all, of the normal lattice contacts are likely to destabilize the virion by aborting formation of the virion as well as by leaving gaps in the virion so that the nucleic acid is not protected. Thus in bacteriophage, unlike the cases of bacteria and spores, it is generally important to retain in the peptide fusion proteins those residues of the coat protein that interact with other proteins in the virion. For example, when using the M13 cpVIII protein, the entire mature protein will generally be retained with the peptide fragment being added to the N-terminus of cpVIII, while on the other hand it may suffice to retain only the last 100 carboxy terminal residues (or even fewer) of the M13 cpIII coat protein in the peptide fusion protein.

Under the appropriate induction, the test peptide library is expressed and exported, as part of the fusion protein, to the bacterial cytoplasm, such as when the λ phage is employed. The induction of the fusion protein(s) may be delayed until some replication of the phage genome, synthesis of some of the phage structural-proteins, and assembly of some phage particles has occurred. The assembled protein chains then interact with the phage particles via the binding of the anchor protein on the outer surface of the phage particle. The cells are lysed and the phage bearing the library-encoded test peptide (that corresponds to the specific library sequences carried in the DNA of that phage) are released and isolated from the bacterial debris.

To enrich for and isolate phage particles that encode a selected test peptide based on binding to a particular progenitor cell population, and thus to isolate the nucleic acid sequence encoding the selected test peptide, phage harvested from the bacterial debris may be affinity purified. As described below, when a test peptide that specifically binds a particular target progenitor cell is desired, the target progenitor cell may be used to retrieve phage displaying the desired test peptide. The phage so obtained may then be amplified by re-infecting host cells. Additional rounds of affinity enrichment followed by amplification may be employed until the desired level of enrichment is reached. Alternatively, or in combination, the amplification may be by nucleic acid amplification, for example by polymerase chain reaction or other similar enzymatic or chemical method of amplification.

### Filamentous Phage

In certain embodiments, the display library is generated using filamentous bacteriophage. Filamentous bacteriophages, which include M13, f1, fd, If1, Ike, Xf, Pf1, and Pf3, are a group of related viruses that infect bacteria. They are termed filamentous because they are long, thin particles comprised of an elongated capsule that envelopes the deoxyribonucleic acid (DNA) that forms the bacteriophage genome. The F pili filamentous bacteriophage (Ff phage) infect only gram-negative bacteria by specifically adsorbing to the tip of F pili, and include fd, f1 and M13.

Compared to other bacteriophage, filamentous phage in general are attractive and M13 in particular is especially attractive because: (i) the 3-D structure of the virion is known; (ii) the processing of the coat protein is well understood; (iii) the genome is expandable; (iv) the genome is small; (v) the sequence of the genome is known; (vi) the virion is physically resistant to shear, heat, cold, urea, guanidinium chloride, low pH, and high salt; (vii) the phage is a sequencing vector so that sequencing is especially easy; (viii) antibiotic-resistance genes have been cloned into the genome with predictable results (Hines et al., Gene 11:207-218, 1980); (ix) it is easily cultured and stored, with no unusual or expensive media requirements for the infected cells, (x) it has a high burst size, each infected cell yielding 100 to 1000 M13 progeny after infection; and (xi) it is easily harvested and concentrated (Salivar et al., Virology 24:359-371, 1964). The entire life cycle of the filamentous phage M13, a common cloning and sequencing vector, is well understood. The genetic structure of M13 is well known, including the complete sequence (Schaller et al. in The Single-Stranded DNA Phages eds. Denhardt et al. (NY: CSHL Press, 1978)), the identity and function of the ten genes, and the order of transcription and location of the promoters, as well as the physical structure of the virion (Smith et al., Science 228:1315-1317, 1985; Raschad et al., Microbiol. Dev. 50:401-427, 1986; Kuhn et al., Science 238:1413-1415, 1987; Zimmerman et al., J. Biol. Chem. 257:6529-6536, 1982; and Banner et al., Nature 289:814-816, 1981). Because the genome is small (6423 bp), cassette mutagenesis is practical on RF M13 (Current Protocols in Molecular Biology, eds. Ausubel et al. (NY: John Wiley & Sons, 1991)), as is single-stranded oligonucleotide directed mutagenesis (Fritz et al. in DNA Cloning, ed by Glover (Oxford, UK: IRC Press, 1985)). M13 is a plasmid and transformation system in itself, and an ideal sequencing vector. M13 can be grown on Rec-strains of E. coli. The M13 genome is expandable (Messing et al. in The Single-Stranded DNA Phages, eds Denhardt et al (NY: CSHL Press, 1978) pages 449-453; and Fritz et al., supra) and M13 does not lyse cells. Extra genes may be inserted into M13 and will be maintained in the viral genome in a stable manner.

The mature capsule or Ff phage is comprised of a coat of five phage-encoded gene products: cpVIII, the major coat protein product of gene VIII that forms the bulk of the capsule; and four minor coat proteins, cpIII and cpIV at one end of the capsule and cpVII and cpIX at the other end of the capsule. The length of the capsule is formed by 2500 to 3000 copies of cpVIII in an ordered helix array that forms the characteristic filament structure. The gene III-encoded protein (cpIII) is typically present in 4 to 6 copies at one end of the capsule and serves as the receptor for binding of the phage to its bacterial host in the initial phase of infection. For detailed reviews of Ff phage structure, see Rasched et al., Microbiol. Rev. 50:401-427, 1986; and Model et al. in The Bacteriophages, Volume 2, R. Calendar, Ed., Plenum Press, pp. 375-456 (1988).

The phage particle assembly involves extrusion of the viral genome through the host cell's membrane. Prior to extrusion, the major coat protein cpVIII and the minor coat protein cpIII are synthesized and transported to the host cell's membrane. Both cpVIII and cpIII are anchored in the host cell membrane prior to their incorporation into the mature particle. In addition, the viral genome is produced and coated with cpV protein. During the extrusion process, cpV-coated genomic DNA is stripped of the cpV coat and simultaneously recoated with the mature coat proteins.

Both cpIII and cpVIII proteins include two domains that provide signals for assembly of the mature phage particle. The first domain is a secretion signal that directs the newly synthesized protein to the host cell membrane. The secretion signal is located at the amino terminus of the polypeptide and targets the polypeptide at least to the cell membrane. The second domain is a membrane anchor domain that provides signals for association with the host cell membrane and for association with the phage particle during assembly. This second signal for both cpVIII and cpIII comprises at least a hydrophobic region for spanning the membrane.

The 50 amino acid mature gene VIII coat protein (cpVIII) is synthesized as a 73 amino acid precoat (Ito et al., Proc. Natl. Acad. Sci. USA 76:1199-1203, 1979). cpVIII has been extensively studied as a model membrane protein because it can integrate into lipid bilayers such as the cell membrane in an asymmetric orientation with the acidic amino terminus toward the outside and the basic carboxy terminus toward the inside of the membrane. The first 23 amino acids constitute a typical signal-sequence which causes the nascent polypeptide to be inserted into the inner cell membrane. An E. coli signal peptidase (SP-I) recognizes amino acids 18, 21, and 23, and, to a lesser extent, residue 22, and cuts between residues 23 and 24 of the precoat (Kuhn et al., J. Biol. Chem. 260:15914-15918, 1985; and Kulu et al., J. Biol. Chem. 260:15907-15913, 1985). After removal of the signal sequence, the amino terminus of the mature coat is located on the periplasmic side of the inner membrane; the carboxy terminus is on the cytoplasmic side. About 3000 copies of the mature coat protein associate side-by-side in the inner membrane.

The sequence of gene VIII is known, and the amino acid sequence can be encoded on a synthetic gene. Mature gene VIII protein makes up the sheath around the circular ssDNA. The gene VIII protein may be a suitable anchor protein because its location and orientation in the virion are known (Banner et al., Nature 289:814-816, 1981). Preferably, the peptide is attached to the amino terminus of the mature M13 coat protein to generate the phage display library. As set out above, manipulation of the concentration of both the wild-type cpVIII and Ab/cpVIII fusion in an infected cell may be utilized to decrease the avidity of the display and thereby enhance the detection of high affinity peptides directed to the target(s).

Another vehicle for displaying the peptide is by expressing it as a domain of a chimeric gene containing part or all of gene III, e.g., encoding cpIII. When monovalent displays are required, expressing the peptide as a fusion protein with cpIII may be a preferred embodiment, as manipulation of the ratio of wild-type cpIII to chimeric cpIII during formation of the phage particles may be readily controlled. This gene encodes one of the minor coat proteins of M13. Genes VI, VII, and IX also encode minor coat proteins. Each of these minor proteins is present in about 5 copies per virion and is related to morphogenesis or infection. In contrast, the major coat protein is present in more than 2500 copies per virion. The gene VI, VII, and 1× proteins are present at the ends of the virion; these three proteins are not posttranslationally processed (Rasched et al., Ann. Rev. Microbiol. 41:507-541, 1986). In particular, the single-stranded circular phage DNA associates with about five copies of the gene III protein and is then extruded through the patch of membrane-associated coat protein in such a way that the DNA is encased in a helical sheath of protein (Webster et al. in The Single-Stranded DNA Phages, eds Dressler et al. (NY:CSHL Press, 1978).

Manipulation of the sequence of cpIII has demonstrated that the C-terminal 23 amino acid residue stretch of hydrophobic amino acids normally responsible for a membrane anchor function can be altered in a variety of ways and retain the capacity to associate with membranes. Ff phage-based expression vectors were first described in which the cpIII amino acid residue sequence was modified by insertion of polypeptide “targets” (Parmely et al., Gene 73:305-318, 1988; and Cwirla et al., Proc. Natl. Acad. Sci. USA 87:6378-6382, 1990) or an amino acid residue sequence defining a single chain peptide domain (McCafferty et al., Science 348:552-554, 1990). It has been demonstrated that insertions into gene III may result in the prodution of novel protein domains on the virion outer surface. (Smith, Science 228:1315-1317, 1985; and de la Cruz et al., J. Biol. Chem. 263:4318-4322, 1988). The peptide gene may be fused to gene III at the site used by Smith and by de la Cruz et al., at a codon corresponding to another domain boundary or to a surface loop of the protein, or to the amino terminus of the mature protein.

Generally, the successful cloning strategy utilizing a phage coat protein, such as cpIII of filamentous phage fd, will provide expression of a peptide chain fused to the N-terminus of a coat protein (e.g., cpIII) and transport to the inner membrane of the host where the hydrophobic domain in the C-terminal region of the coat protein anchors the fusion protein in the membrane, with the N-terminus containing the peptide chain protruding into the periplasmic space.

Similar constructions could be made with other filamentous phage. Pf3 is a well known filamentous phage that infects Pseudomonos aerugenosa cells that harbor an IncP-I plasmid. The entire genome has been sequenced (Luiten et al., J. Virol. 56:268-276, 1985) and the genetic signals involved in replication and assembly are known (Luiten et al., DNA 6:129-137, 1987). The major coat protein of PF3 is unusual in having no signal peptide to direct its secretion. The sequence has charged residues ASP-7, ARG-37, LYS-40, and PHE44 which is consistent with the amino terminus being exposed. Thus, to cause a peptide to appear on the surface of Pf3, a tripartite gene may be constructed that comprises a signal sequence known to cause secretion in P. aerugenosa, fused in-frame to a gene fragment encoding the peptide sequence, that is fused in-frame to DNA encoding the mature Pf3 coat protein. Optionally, DNA encoding a flexible linker of one to 10 amino acids is introduced between the peptide gene fragment and the Pf3 coat-protein gene. This tripartite gene is introduced into Pf3 so that it does not interfere with expression of any Pf3 genes. Once the signal sequence is cleaved off, the peptide is in the periplasm and the mature coat protein acts as an anchor and phage-assembly signal.

### Bacteriophage ΦX174

The bacteriophage ΦX174 is a very small icosahedral virus that has been thoroughly studied by genetics, biochemistry, and electron microscopy (see The Single Stranded DNA Phages (eds. Denhardt et al. (NY:CSHL Press, 1978)). Three gene products of ΦX174 are present on the outside of the mature virion: F (capsid), G (major spike protein, 60 copies per virion), and H (minor spike protein, 12 copies per virion). The G protein comprises 175 amino acids, while H comprises 328 amino acids. The F protein interacts with the single-stranded DNA of the virus. The proteins F, G, and H are translated from a single mRNA in the viral infected cells. As the virus is so tightly constrained because several of its genes overlap, ΦX174 is not typically used as a cloning vector due to the fact that it can accept very little additional DNA. However, mutations in the viral G gene (encoding the G protein) may be rescued by a copy of the wild-type G gene carried on a plasmid that is expressed in the same host cell (Chambers et al., Nucleic Acids Res. 10:6465-6473, 1982). In one embodiment, one or more stop codons may be introduced into the G gene so that no G protein is produced from the viral genome. The variegated peptide gene library may then be fused with the nucleic acid sequence of the H gene. An amount of the viral G gene equal to the size of peptide gene fragment is eliminated from the ΦX174 genome, such that the size of the genome is ultimately unchanged. Thus, in host cells also transformed with a second plasmid expressing the wild-type G protein, the production of viral particles from the mutant virus is rescued by the exogenous G protein source. Where it is desirable that only one test peptide be displayed per φX174 particle, the second plasmid may further include one or more copies of the wild-type H protein gene so that a mix of H and test peptide/H proteins will be predominated by the wild-type H upon incorporation into phage particles.

### Large DNA Phage

Phage such as λ or T4 have much larger genomes than do M13 or ΦX174, and have more complicated 3-D capsid structures than M13 or ΦX174, with more coat proteins to choose from. In embodiments of the invention whereby the test peptide library is processed and assembled into a functional form and associates with the bacteriophage particles within the cytoplasm of the host cell, bacteriophage λ and derivatives thereof are examples of suitable vectors. The intracellular morphogenesis of phage λ may potentially prevent protein domains that ordinarily contain disulfide bonds from folding correctly. However, variegated libraries expressing a population of functional peptides, which include such bonds, have been generated in λ phage. Examples of λ phage display libraries include Maruyama et al., Proc. Natl. Acad. Sci. USA 91:8273-8277, 1994; Sternberg et al., Proc. Natl. Acad. Sci. USA 92(5):1609-13, 1995. Such strategies take advantage of the rapid construction and efficient transformation abilities of λ phage. There are also commercial kits available that may be readily adapted to generate the display libraries contemplated herein.

When used for expression of peptide sequences, exogenous nucleotide sequences may be readily inserted into a λ vector. For instance, variegated peptide libraries may be constructed by modification of λ ZAP II through use of the multiple cloning site of a λ ZAP II vector (Huse et al. supra).

Other examples of phage particles useful for purposes of the instant invention include T7, P2, P4, MS2, and f2.

### Bacterial Cells as Display Packages

Recombinant peptides are able to cross bacterial membranes after the addition of appropriate secretion signal sequences to the N-terminus of the protein (Better et al., Science 240:1041-1043, 1988; and Skerra et al., Science 240:1038-1041, 1988). In addition, recombinant peptides have been fused to outer membrane proteins for surface presentation. For example, one strategy for displaying peptides on bacterial cells comprises generating a fusion protein by inserting the peptide into cell surface exposed portions of an integral outer membrane protein (Fuchs et al., Bio/Technology 9:1370-1372, 1991). In selecting a bacterial cell to serve as the display package, any well-characterized bacterial strain will typically be suitable, provided the bacteria may be grown in culture, engineered to display the test peptide library on its surface, and is compatible with the particular affinity selection process practiced in the subject method. Among bacterial cells, the preferred display systems include Salmonella typhirnurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. Many bacterial cell surface proteins useful in the present invention have been characterized, and works on the localization of these proteins and the methods of determining their structure include Benz et al., Ann. Rev. Microbiol. 42: 359-393, 1988; Balduyck et al., Biol Chem Hoppe-Seyler 366:9-14, 1985; Ehrmann et al., Proc. Natl. Acad. Sci. USA 87:7574-7578, 1990; Heijne et al., Protein Engineering 4:109-112, 1990; Ladner et al. U.S. Pat. No. 5,223,409; Ladner et al. PCT International Publication No. WO88/06630; Fuchs et al., Bio/technology 9:1370-1372, 1991; and Goward et al., TIBS 18:136-140, 1992.

To further illustrate, the LamB protein of E. coli is a well understood surface protein that may be used to generate a variegated library of test peptides on the surface of a bacterial cell (see, for example, Ronco et al., Biochemie 72:183-189, 1990; van der Weit et al., Vaccine 8:269-277, 1990; Charabit et al., Gene 70:181-189, 1988; and Ladner, U.S. Pat. No. 5,222,409). LamB of E. coli is a porin for maltose and maltodextrin transport, and serves as the receptor for adsorption of bacteriophages λ and K10. LamB is transported to the outer membrane if a functional N-terminal signal sequence is present (Benson et al., Proc. Natl. Acad. Sci. USA 81:3830-3834, 1984). As with other cell surface proteins, LamB is synthesized with a typical signal-sequence which is subsequently removed. Thus, the variegated peptide gene library may be cloned into the LamB gene such that the resulting library of fusion proteins comprise a portion of LamB sufficient to anchor the protein to the cell membrane with the test peptide fragment oriented on the extracellular side of the membrane. Secretion of the extracellular portion of the fusion protein may be facilitated by inclusion of the LamB signal sequence, or other suitable signal sequence, as the N-terminus of the protein.

The E. coli LamB has also been expressed in functional form in S. typhimurium (Harkki et al., Mol. Gen. Genet. 209:607-611, 1987), V cholerae (Harkki et al., Microb. Pathol. 1:283-288, 1986), and K. pneumonia (Wehmeier et al., Mol. Gen. Genet. 215:529-536, 1989), so that one could display a population of test peptides in any of these species as a fusion to E. coli LamB. Moreover, K. pneumonia expresses a maltoporin similar to LamB which could also be used. In P. aeruginosa, the Dl protein (a homologue of LamB) may be used (Trias et al., Biochem. Biophys. Acta 938:493-496, 1988). Similarly, other bacterial surface proteins, such as PAL, OmpA, OmpC, OmpF, PhoE, pilin, BtuB, FepA, FhuA, IutA, FecA and FhuE, may be used in place of LamB as a portion of the display means in a bacterial cell.

In another exemplary embodiment, the fusion protein may be derived using the FliTrx™ Random Peptide Display Library (Invitrogen). That library is a diverse population of random dodecapeptides inserted within the thioredoxin active-site loop inside the dispensable region of the bacterial flagellin gene (fliC). The resultant recombinant fusion protein (FLITRX) is exported and assembled into partially functional flagella on the bacterial cell surface, displaying the random peptide library.

Peptides are fused in the middle of thioredoxin, therefore, both their N- and C-termini are anchored by thioredoxin's tertiary structure. This results in the display of a constrained peptide. By contrast, phage display proteins are fused to the N-terminus of phage coat proteins in an unconstrained manner. The unconstrained molecules possess many degrees of conformational freedom which may result in the lack of proper interaction with the target molecule. Without proper interaction, potential protein-protein interactions may be missed.

Moreover, phage display is limited by the low expression levels of bacteriophage coat proteins. FliTrx™ and similar methods may overcome this limitation by using a strong promoter to drive expression of the test peptide fusions that are displayed as multiple copies.

According to the present invention, it is contemplated that the FliTrx vector may be modified to provide a vector that is differentially spliced in mammalian cells to yield a secreted, soluble test peptide.

### Bacterial Spores as Display Packages

Bacterial spores also have desirable properties as display package candidates in the subject method. For example, spores are much more resistant than vegetative bacterial cells or phage to chemical and physical agents, and hence permit the use of a great variety of affinity selection conditions. Also, Bacillus spores neither actively metabolize nor alter the proteins on their surface. However, spores have the disadvantage that the molecular mechanisms that trigger sporulation are less well worked out than is the formation of M13 or the export of protein to the outer membrane of E. coli, though such a limitation is not a serious detractant from their use in the present invention.

Bacteria of the genus Bacillus form endospores that are extremely resistant to damage by heat, radiation, desiccation, and toxic chemicals (reviewed by Losick et al., Ann. Rev. Genet. 20:625-669, 1986). This phenomenon is attributed to extensive intermolecular cross-linking of the coat proteins. In certain embodiments of the subject method, such as those including relatively harsh affinity separation steps, Bacillus spores may be the preferred display package. Endospores from the genus Bacillus are more stable than are, for example, exospores from Streptomyces. Moreover, Bacillus subtilis forms spores in 4 to 6 hours, whereas Streptomyces species may require days or weeks to sporulate. In addition, genetic knowledge and manipulation is much more developed for B. subtilis than for other spore-forming bacteria.

Viable spores that differ only slightly from wild-type are produced in B. subtilis even if any one of four coat proteins is missing (Donovan et al., J. Mol. Biol. 196:1-10, 1987). Moreover, plasmid DNA is commonly included in spores, and plasmid encoded proteins have been observed on the surface of Bacillus spores (Debro et al., J. Bacteriol. 165:258-268, 1986). Thus, it may be possible during sporulation to express a gene encoding a chimeric coat protein comprising a peptide of the variegated gene library, without interfering materially with spore formation.

To illustrate, several polypeptide components of B. subtilis spore coat (Donovan et al., J. Mol. Biol. 196:1-10, 1987) have been characterized. The sequences of two complete coat proteins and amino-terminal fragments of two others have been determined. Fusion of the test peptide sequence to cotC or cotd fragments is likely to cause the peptide to appear on the spore surface. The genes of each of these spore coat proteins are preferred as neither cotC or cotD are post-translationally modified (see Ladner et al. U.S. Pat. No. 5,223,409).

### Selection and Identification of Ligands

A variegated ligand display library may be subjected to affinity enrichment in order to select for test ligands that associate with preselected target progenitor cells. The term “affinity separation” or “affinity enrichment” includes creating a sub-population of display packages that has been enriched for those members that have a minimum level of affinity, depending on the stringency of the conditions, for a particular progenitor cell population. Enrichment may be achieved, for example, by panning on live cells or tissue, i.e., the equivalent of affinity chromatography utilizing progenitor cells, or by fluorescence activated cell sorting. In each embodiment, the individual display packages within a display package library are ultimately separated based on the ability of the associated test ligand to associate with the target progenitor cell of interest. See, for examples of affinity enrichment steps than may be adapted for use in the present method, the Ladner et al. U.S. Pat. No. 5,223,409; the Kang et al. International Publication No. WO 92/18619; the Dower et al. International Publication No. WO 91/17271; the Winter et al. International Publication No. WO 92/20791; the Markland et al. International Publication No. WO 92/15679; the Breitling et al. International Publication No. WO 93/01288; the McCafferty et al. International Publication No. WO 92/01047; the Garrard et al. International Publication No. WO 92/09690; and the Ladner et al. International Publication No. WO 90/02809.

In certain preferred embodiments, the ligand display library is first pre-enriched for ligands specific for the target by first contacting the display library with any negative controls or other cell populations for which differential binding, relative to the target progenitor cell, is desired. Subsequently, the non-binding fraction from that pre-treatment step is contacted with the target progenitor cells, and ligands from the display library that are able to specifically associate with the target progenitor cell are isolated.

With respect to affinity chromatography, it will be generally understood by those skilled in the art that a great number of techniques may be adapted for use in the present invention, ranging from batch elution from cultured cells to other biopanning techniques.

The ligand display library may be applied to a sample of cells or tissue that includes at least one target progenitor cell under conditions compatible with the association of a test ligand with the target progenitor cell. Although human cells or tissue are preferred for use in the invention, the cells or tissue to be used according to the invention are not limited to those from human sources. Cells and tissues from other mammalian species including, but not limited to, equine, canine, feline, porcine, bovine, and ovine sources, or rodent species such as mouse or rat, may be used.

The cell or tissue sample will typically include pluripotent stem cells. The sample may include adherent or non-adherent cell cultures, or may be cultured tissue (including fetal tissues such as inner cell mass tissue). The sample may, for example, include cells that have been subjected to analytical reprogramming technology as defined above. The sample may, for example, include hES cells, hEG cells, hED cells, and/or pluripotent stem cells of the first four weeks of human embryonic development, including, but not limited to, pluripotent endodermal, mesodermal, or ectodermal progenitor cells.

In some embodiments, the cell or tissue sample may include cells that have been purified prior to use in the invention, for example, by flow cytometry.

In some embodiments, the cell or tissue sample may include cells that have been subjected to genetic selection prior to or during use in the invention. See, for example, Li et al., Curr. Biol. 8:971-974, 1998. In some embodiments, the cell or tissue sample may include cells derived from a single cell or a small number of similar cells differentiated, or in the process of differentiating, from pluripotent stem cells, as described, for example, in U.S. Patent Application Nos. 60/738,912, filed Nov. 21, 2005, 60/791,400, filed Apr. 11, 2006, and 60/798,103, filed May 4, 2006, the disclosures of which are incorporated herein in their entireties.

As set forth above, a negative control or other cell population for which differential binding, relative to the target progenitor cell, is desired may be used to negatively select (e.g., remove) display packages in order to increase the selectivity of the remaining display packages for the target progenitor cells. The population may then be fractionated by washing with a solute that does not greatly affect specific binding of ligands to cells in the affinity maturation sample, but that disrupts non-specific binding. A certain degree of control may be exerted over the binding characteristics of the ligands recovered from the display library by adjusting the conditions of the binding incubation and subsequent washing. As is understood by those of skill in the art, the temperature, pH, ionic strength, divalent cation concentration, and the volume and duration of the washing may select for ligands within a particular range of affinity and specificity. Selection based on slow dissociation rate, which is usually predictive of high affinity, is a very practical route. Such selection may be done either by continued incubation in the presence of a saturating amount of the affinity maturation cells, or by increasing the volume, number, and/or length of the washes. In each case, the rebinding of dissociated peptide-display package is prevented, and with increasing time, ligand display packages of higher and higher affinity may be recovered. Moreover, additional modifications of the binding and washing procedures may be applied to find ligands with special characteristics. The affinities of some ligands may be dependent on ionic strength or cation concentration. Specific examples are ligands that depend on Ca⁺⁺ for binding activity and that lose or gain binding affinity in the presence of EGTA or other metal chelating agent. Such ligands may be identified in the ligand display library by a double screening technique, wherein display packages that bind the affinity maturation cells in the presence of Ca⁺⁺ are first isolated. Display packages that fail to bind in the presence of EGTA may then be identified.

In some embodiments, specifically bound display packages may be eluted from the affinity maturation cells after a “washing” step to remove non-specifically bound display packages. Elution may be effected, for example, by specific desorption (e.g., by treatment with excess target) or non-specific desorption (e.g., by adjusting pH, varying ionic strength, or using chaotropic agents). In preferred embodiments, the elution protocol does not damage the display package, so that the enriched population of display packages may be identified, for example by amplification. Potential eluants include salts (such as those in which one of the counter ions is Na⁺, NH₄⁺, Rb⁺, SO₄²⁻, H₂PO₄⁻, citrate, K⁺, Li⁺, Cs⁺, HSO₄⁻, CO₃²⁻, Ca²⁺, Sr²⁺, Cl⁻, PO₄²⁻, HCO₃⁻, Mg₂⁺, Ba₂⁺, Br⁻, HPO₄²⁻, or acetate), acid, heat, and, when available, soluble forms of the target (or analogs thereof). Because bacteria continue to metabolize during the affinity separation step and are generally more susceptible to damage by harsh conditions, the choice of buffer components (especially eluants) may be more restricted when the display package is a bacteria rather than the other types of display packages. Neutral solutes, such as ethanol, acetone, ether, or urea, are examples of other agents useful for eluting the bound display packages.

In some embodiments, the specifically bound display packages show similar affinity to the target progenitor cell and to other cells in the sample. In other embodiments, the specifically bound display packages are less selective for the target progenitor cell than for the other cells in the sample. In preferred embodiments, the specifically bound display packages are more selective for the target progenitor cell than the other cells in the sample.

In some embodiments, the display packages associated with target progenitor cells may remain bound to the external surface of the cells during subsequent incubations. In other embodiments, the display packages associated with target progenitor cells may be internalized by receptor-mediated endocytosis during such incubations.

In certain embodiments, display packages that are specifically associated with target cells need not be eluted from the cells prior to their identification, but rather, the cell-bound display packages may be used directly to inoculate a suitable growth media for amplification.

In preferred embodiments, affinity enriched display packages are iteratively amplified and subjected to further rounds of affinity separation until enrichment of the desired specificity for the target progenitor cell is detected.

Where the display package is a phage particle, the fusion protein generated with the coat protein may interfere substantially with the subsequent amplification of eluted phage particles, particularly in embodiments wherein the cpIII protein is used as the display anchor. Even though present in only one of the 5-6 tail fibers, some peptide constructs because of their size and/or sequence, may cause severe defects in the infectivity of their carrier phage. This may cause a loss of phage from the population during reinfection and amplification following each cycle of panning. In one embodiment, the peptide may be expressed on the surface of the display package so as to be susceptible to proteolytic cleavage of the covalent linkage of at least the target binding sites of the displayed peptide from the remaining package. For instance, where the cpIII coat protein of M13 is employed, such a strategy may be used to obtain infectious phage by treatment with an enzyme that cleaves between the test peptide portion and cpIII portion of a tail fiber fusion protein (e.g. such as the use of an enterokinase cleavage recognition sequence).

To further minimize problems associated with defective or lost infectivity, DNA prepared from the eluted phage may be transformed into host cells by electroporation or well known chemical means. The cells may be cultivated for a period of time sufficient for marker expression, and selection may then be applied as typically done for DNA transformation. The colonies may be amplified, and phage particles harvested for a subsequent round(s) of panning.

In preferred embodiments of the invention, the methods may be used to identify ligands that are associated with target progenitor cells at various stages of proliferation, differentiation, and/or apoptosis. The target progenitor cells are preferably allowed to proliferate, differentiate, and/or undergo apoptosis for at least 1 day after contacting the ligand display library with the target progenitor cell. In more preferable embodiments, the target progenitor cells are allowed to proliferate, differentiate, and/or undergo apoptosis for at least 2 days after contacting the display packages with the target progenitor cell. In even more preferable embodiments, the target progenitor cells are allowed to proliferate, differentiate, and/or undergo apoptosis for at least 4 days, at least 6 days, at least 12 days, at least 18 days, or even longer after contacting the display packages with the target progenitor cell.

In some embodiments of the invention, the target progenitor cells are allowed to proliferate, differentiate, and/or undergo apoptosis prior to contacting the ligand display library with the target progenitor cell. In specific embodiments, the target progenitor cells are allowed to proliferate, differentiate, and/or undergo apoptosis for at least 1 day prior to contacting the display packages with the target progenitor cell. In more specific embodiments, the target progenitor cells are allowed to proliferate, differentiate, and/or undergo apoptosis for at least 2 days, at least 4 days, at least 6 days, at least 12 days, at least 18 days, or even longer prior to contacting the display packages with the target progenitor cell. In some embodiments of the invention, the target progenitor cells are treated with an agent that affects cell growth or metabolism during the period either prior to or after contacting the target progenitor cells with the ligand display library. Examples of such agents include agents that affect cell proliferation, cell differentiation, cell death, intracellular calcium mobilization, intracellular protein phosphorylation, phospholipid metabolism, expression of cell-specific marker genes, etc. See also below. In certain specific embodiments, one or more of the ligands in the display library may themselves affect cell growth or metabolism upon binding to a target progenitor cell. Such ligands may, for example, induce or inhibit cell proliferation, cell differentiation, and/or cell death. In some embodiments, one or more of the ligands in the display library may, for example, induce or inhibit changes in intracellular calcium mobilization, intracellular protein phosphorylation, phospholipid metabolism, and/or expression of cell-specific marker genes. It should be understood that the term “inhibit” embraces both the partial loss of a specified activity as well as the complete loss of that activity.

In certain embodiments, the target progenitor cell includes a reporter gene construct containing a reporter gene in operative linkage with one or more transcriptional regulatory elements responsive to the binding or the ligand, or responsive to changes in phenotype of the cell as a consequence thereto. For instance, the reporter gene may encode a gene product that gives rise to a detectable signal selected from the group consisting of color, fluorescence, luminescence, cell viability, relief of a cell nutritional requirement, cell growth, and drug resistance.

In some embodiments of the invention, ligand display libraries are used to present candidate ligands to tissue isolated from animal that contains stem cells, including embryonic, fetal, and adult tissues, as well as teratomas, in order to identify ligands that specifically associate with such specific differentiated cell types.

In other embodiments, the ligand display libraries are used to identify candidate ligands that specifically associate with cells that differentiate in vitro from pluripotent stem cells such as ES and ED cells.

In still other embodiments, the ligand display libraries are used to identify candidate ligands that specifically associate with cells during differentiation, for example as induced or inhibited by culture conditions or ectopic agents. For instance, the subject method may be used to identify ligands that selectively bind to cells following treatment with inducers and differentiation agents such as growth factors, cytokines, extracellular matrix components, nucleic acids encoding the foregoing, steroids, and morphogens or neutralizing antibodies to such factors. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3.

Differentiation agents according to the invention also include growth factors such as 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropioetin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocytemacrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thromopoietin, transforming growth factor-(alpha, beta-1,2,3,4,5), tumor necrosis factor (alpha and beta), vascular endothelial growth factors, bone morphogenic proteins, ascorbic acid, and retinoic acid.

Differentiation agents according to the invention also include hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin.

In addition, differentiation agents according to the invention include extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan.

Differentiation agents according to the invention also include antibodies to the previously-mentioned cytokines, growth factors, hormones, and extracelluar matrix components, and their receptors.

### Ligands

Another aspect of the present invention provides ligands identified by the methods of the invention and having a desired binding specificity and/or affinity for a target progenitor cell or a component thereof. Such ligands may be capable of regulating a biological process in a target cell.

The ligands identified using the above methods may therefore be useful as markers, alone or in conjunction with a detectable label, to identify reagents and conditions that have an effect on the proliferation, differentiation, and/or viability of desired cell types. Such ligands may also be used in the preparation of a pure population of the targeted progenitor cells or to eliminate specific cell types from a mixture of cell types (such as through affinity separation or selective delivery of toxins).

Uses for ligands discovered by selection from such ligand display libraries on proliferating, differentiating, and/or apoptosing cells (e.g., ES cells) include, but are not limited to:
Affinity-purification of progenitors and expansion of differentiated cells that are candidates for cell therapy.
Development of assays for optimization of culture conditions for differentiation and expansion of progenitor cells.
Imaging of progenitor cells in therapeutic models and as part of therapeutic protocols.
Development of new and novel cell proliferation and/or differentiation agents.

In certain instances, the ligands identified by the subject method may have direct biological activity on the cells with which they are contacted, such as inducing or inhibiting differentiation, inducing or inhibiting proliferation, improving viability, or selectively killing, such as, for example, by apoptosis.

The subject invention also specifically contemplates that peptide ligands identified according to the instant invention be converted into peptidomimietics, e.g., by replacement of backbone or sidechain moieties with non-naturally occurring analogs.

Moreover, in certain embodiments, the subject invention includes the formulation, with a pharmaceutically acceptable carrier, of one or more test ligands capable of regulating a biological process in the target progenitor cell, or mimetics thereof.

In some embodiments, identification of a ligand for a target progenitor cell may identify a connection between the ligand and a signaling pathway within the cell or its neighbors. For example, ligands containing an RXXR motif may mimic one or more peptide hormones that are normally processed by Furin or other members of the proprotein convertase (PC) family of proteases that process many prohormones and growth factors.

Another aspect of this invention is to identify surface bound ligands that will stimulate ES cells to differentiate along defined lineages, or alternatively, to retain their stemness under particular culture conditions. For instance, the subject display libraries may be presented in a form bound or otherwise associated with a solid surface in order to create an artificial microenvironment for cell attachment and growth. Stem cells may be engineered to express a detectable reporter gene when differentiated along a particular lineage pathway. To further illustrate, a phage display library, amplified phage clones or pooled clones may be attached to tissue culture plastic and cells may be plated and allowed to grow over the phage particles. Phage clones may be arrayed on a single plate or in multi-well plates. The appearance of reporter gene expression indicates the presence of ligands that induce differentiation. Phage particles are removed from the plate in the area occupied by reporter gene expressing cells and amplified by bacterial infection or DNA amplification. Alternatively, host bacteria may be added to the plate in the region of reporter expressing cells and phage may be amplified by infection in-situ. The structure of the differentiation-inducing ligand may be determined by sequencing the selected phage DNA. Multiple rounds of selection on solid surface-bound phages may be performed as in standard phage display.

In other embodiments of the invention, the ligand display libraries are used to identify candidate ligands that specifically bind cells during proliferation and/or apoptosis, for example, as induced or inhibited by culture conditions or ectopic agents. For instance, the subject method may be used to identify ligands that selectively bind to cells following treatment with agents inducing or inhibiting proliferation and/or apoptosis, such as FGF, EGF, TGF, PDGF, IFN, NGF, insulin, actinin, pentapeptide growth inhibitor, interleukins, GM-CSF, G-CSF, TNF, IGF, etc.

### Cells

Another aspect of the present invention provides cells identified by the methods of the invention. In some embodiments, the cells are target progenitor cells that selectively bind at least one ligand of the invention. In some embodiments, the cells are identified following their differentiation in the presence of the ligand display library. As already described a target progenitor cell may be allowed to differentiate either before or after it is contacted with the display library. The differentiated target cell may in some embodiments be identified because it binds at least one display package. In some embodiments, the display package is labeled, such as, for example, by an identifiable marker or label. Differentiated cells with bound display packages may in some embodiments be identified and isolated using the bound marker or label. The identifiable marker or label may be, for example, a radioactive or fluorescent label, as is well understood in the art. In specific embodiments, the display packages are labeled with quantum dots. In some embodiments, the display package is selectively bound to the target differentiated cell.

In some embodiments, the differentiated cells may contain a specific surface marker that allows them to be separated from surrounding cells using a specific antibody and fluorescent or magnetic cell sorting (FACS or MACS) prior to the identification of a bound display package. In other embodiments, the differentiated cells are engineered to express a cell-specific reporter gene that allows the cells to be isolated following proliferation, differentiation, and/or apoptosis. Examples of such reporter genes include, for example, green fluorescent protein (GFP) and its variants. Subject cells are readily isolated by FACs following their incubation with the ligand display library. Cells may alternatively be engineered to express a cell-specific selectable gene, such as a gene that provides drug resistance, e.g., the neo gene. Subject cells may be isolated by selecting for growth on the drug following their incubation with the ligand display library. Those of skill in the art would readily understand the use of such techniques.

### Testing of Selected Ligands

The test ligands identified using the methods of the instant invention may, in some cases, be further tested for activity. In some embodiments, the identified test ligands will be tested following their chemical synthesis. In other embodiments, the test ligands will be generated directly from the isolated display package.

In one embodiment, the display library is a peptide library that can be shifted to a “secretion mode”. In the secretion mode, the peptide library, which was enriched and identified in the display mode, is transfected into and expressed by eukaryotic cells. In this mode, the test peptides are secreted by the host cells and screened for biological activity on the target progenitor cells.

To further illustrate, the library vectors may be constructed to include eukaryotic splice sites such that, in the mature mRNA, elements required for the display mode in prokaryotic cells are spliced out—at least those elements which would interfere with the secretion mode. A variety of naturally and non-naturally occurring splice sites are available in the art and can be selected for, e.g., optimization in particular eukaryotic cells selected.

In such embodiments, the vectors are selected so as to also be used to transfect a cell that can be co-cultured with a target progenitor cell. A biologically active protein secreted by the host cell will diffuse to neighboring target progenitor cells and induce a particular biological response, such, as to illustrate, proliferation or differentiation, or activation of a signal transduction pathway that is directly detected by other phenotypic criteria. The pattern of detection of biological activity will resemble a gradient function, and will allow the isolation (generally after several repetitive rounds of selection) of cells producing peptides having certain activity on the target progenitor cell. Likewise, antagonists of a given factor may be selected in similar fashion by the ability of the host cell producing a functional antagonist to protect neighboring target progenitor cells from the effect of exogenous factor added to the culture media.

To further illustrate, target progenitor cells may be cultured in 24-well microtitre plates. Host cells are transfected with the affinity matured peptide library, recovered after the display mode step, and cultured in cell culture inserts (e.g Collaborative Biomedical Products, Catalog #40446) that are able to fit into the wells of the microtitre plate. The cell culture inserts are placed in the wells such that recombinant test peptides secreted by the cells in the insert can diffuse through the porous bottom of the insert and contact the target progenitor cells in the microtitre plate wells. After a period of time sufficient for a secreted test peptide to produce a measurable response in the target progenitor cells, the inserts are removed and the effect of the peptides on the target progenitor cells determined. For example, where the activity desired from the test peptides is the induction of neuronal differentiation, then fluorescently-labeled antibodies specific for Islet-1 or other neuronal markers may be used to score for induction in the target cells as indicative of a functional neurotrophic peptide in that well. Cells from the inserts corresponding to wells which score positive for activity may be split and re-cultured on several inserts, the process being repeated until the active peptide is identified.

When screening for bioactivity of test peptides, intracellular second messenger generation may be measured directly. For instance, a variety of intracellular effectors have been identified as being receptor- or ion channel-regulated, including adenylyl cyclase, cyclic GMP, phosphodiesterases, phosphoinositidases, phosphoinositol kinases, and phospholipases, as well as a variety of ions. In still another embodiment, a heterologous reporter gene construct may be used to provide the function of an indicator gene. Reporter gene constructs are prepared by operatively linking a reporter gene with at least one transcriptional regulatory element. If only one transcriptional regulatory element is included it must be a regulatable promoter. At least one of the selected transcriptional regulatory elements must be indirectly or directly regulated by the activity of the selected cell-surface receptor whereby activity of the receptor can be monitored via transcription of the reporter genes.

Suitable host cells for use in the secretion mode include prokaryotes, yeast, or higher eukaryotic cells, including plant and animal cells, especially mammalian cells, that can be co-cultured with the target progenitor cell. Prokaryotes include gram negative or gram positive organisms. Examples of suitable mammalian host cell lines include the COS-7 line of monkey kidney cells (ATCC CRL 1651) (Gluzman (1981) Cell 23:175) CV-1 cells (ATCC CCL 70), L cells, C127, 3T3, Chinese hamster ovary (CHO), HeLa, HEK-293, SWISS 3T3, and BHK cell lines.

### Additional Uses of the Methods

It is envisioned that the disclosed methods for the identification of novel ligands to pluripotent stem cells such as ES and ED cells and cells derived from these cells will have application in identifying conditions that lead to the differentiation of particular cell types, for the purification of desired cell types from a mixture of cell types, and for eliminating particular undesired cell types from a mixture of cell types.

Because of the flexibility of the system, the subject method may be used in a broad range of applications, including, as described above, for the selection of ligands having effects on proliferation, differentiation, and cell death. Ligands having effects on cell migration and other cellular properties may also be selected according to the subject method. Such effects may include, as described above, either induction or inhibition of the property.

The invention now being generally described, it will be more readily understood by reference to the following examples which are included merely for purposes of illustration of certain aspects and embodiments of the present invention, and are not intended to limit the invention.

### EXAMPLES

### Example 1

### Identification of Ligands that Bind Differentiation Antigens Using Gene Trap-Based Selection

Description: The gene trap method is used to tag cells by means of a genetic marker that are at various stages of differentiation between a pluripotent stem cell and a fully differentiated cell. Gene trapped cells are selected by virtue of a genetic marker such as a fluorescent protein or drug resistance gene. The gene trapped cells display one or more differentiation antigens on their surface that are characteristic of the differentiation status of the cell. Ligands that bind the differentiation antigens are selected from large libraries of ligands displayed on filamentous phage particles by means of reiterative cycles of contacting the cells with the library, removal of unbound phage and recovery of binding phage. Each cycle enriches the library for cell binding ligands. Alternatively, bound phage are recovered at an early selection cycle and individual phage are amplified, prepared and screened directly in multi-well plate format for reactivity with the gene trapped cells of interest. Phage displaying ligands that bind the gene trapped cells are further screened for specificity on cultured stem cells or mammalian embryos, or on tissues that contain stem cells such as teratoma, fetal and adult tissues, to identify ligands that bind differentiation antigens that are characteristic of various cell lineages at various stages of differentiation.
Methods: Standard methods are used to prepare an ligand phage display library (Hoogenboom et al., Immunotechnology 4(1):1-20, 1998.). Specifically, antibody libraries are prepared from a suitable animal such as mouse, rat, rabbit, chicken or a pool of human spleen mRNA. For animal derived antibody libraries, the animal may be immunized with the selected gene-trapped cells or immunologically naïve animals may be used. The GFP expressing gene trapped ES cells are contacted with the ligand display library and allowed to bind for 1-3 hours at room temperature. In some instances longer incubation times (up to 72 hours) may be used to bias the library towards internalized ligands. Unbound phage particles are removed from the cells using repeated washing (10 to 20 times) in PBS (phosphate buffered saline) and exposure to low pH 2.0 or other methods described previously (Barry et al., Nat. Med. 2(3):299-305, 1996; Kassner et al., Biochem. Biophys. Res. Commun., 264(3):921-8, 1999). The phage particles displaying ligands that bind to GFP expressing gene trapped cells are recovered by infecting a suitable host bacteria directly with lysates prepared from the sorted cells. Alternatively, the phage are recovered by amplification of phage DNA from the cell lysates using PCR (Kassner et al., Biochem. Biophys. Res. Commun. 264(3):921-8, 1999) or RCA (Burg et al., DNA Cell Biol. 23(7):457-62, 2004) and subsequent re-cloning of the ligand encoding DNA into the phage vector. The selection is repeated until the library is reduced sufficiently in complexity for sequence analysis and screening of individual ligands from the selected pool. Additional experiments may be performed where the subsequent selection steps are omitted and the ligands screened directly for binding to stem cells or histological sections from mammalian embryos, or on tissues that contain stem cells such as teratoma, fetal and adult tissues.

The initial selections of ligand libraries on gene trapped stem cells may result in a preponderance of ligands that bind the most highly abundant cell surface antigens. Once the ligands to the highly abundant antigens have been isolated, the free ligands (not bound to a phage) are used as a blocking agent to prevent reselection of phage displayed ligands against highly abundant antigens. In this way, the selection is now biased away from ligands that bind highly abundant antigens and towards those that bind less abundant antigens.

In the above example, the gene trapped cells are incubated with the phage library after they have been selected by cell sorting or drug resistance. Ligands that bind differentiation antigens may also be selected by incubation of the ligand display library with the stem cells prior to selection of the gene trap marker. In this case, the stem cells are transfected with the gene trap vector and incubated under conditions known to stimulate differentiation. The cells are monitored for expression of the gene trap tag (e.g. GFP). The cells are incubated with the ligand display library, unbound phages are removed and the cells are sorted for GFP expressing cells by FACs. Phage that bound to the GFP expressing cells are recovered from the sorted cell population and characterized by sequence analysis, and used for a subsequent selection round if necessary. Individual ligand display phage particles are screened for specificity against differentiation antigens on cultured stem cells and mammalian embryos.

### Example 2

### Identification of Ligands/Ligands that Bind Differentiation Antigens using Selection of Phage Display Libraries in Morphological Structures in Developing Primate Embryos

Description: Cells of distinct morphology can be identified within the developing embryos of mammals starting with the appearance of the primitive streak through to the early fetus. The distinct cells are precursors or progenitors of differentiated tissues however little is known about what distinguish them at the molecular level and in particular what cell surface molecules are present. The distinguishing cell surface antigens must also appear on certain progenitor cells in populations of cultured human ES cells that can under appropriate conditions form lineages of cells leading up to differentiated tissues (i.e. exposure to certain growth factors in culture or the formation of teratomas in-vivo). In this example, ligand libraries displayed on phage are selected against a sample of tissue taken from a primate embryo using microdissection. Selections of the library against cultured stem cells may also be included to bias the library towards ligands that bind antigens that are shared between a subpopulation of the cultured stem cells and the target tissue within the primate embryo. Selected ligands against differentiation antigens are used to identify and purify progenitor cells in stem cell cultures.

Methods: Morphologically distinct cells in primate embryonic tissues are isolated using known methods of microdissection (Nawshad et al., Dev. Dyn. 230(3):529-34, 2004). The tissues are selected from primate (Rhesus or Cynomologous) embryos at various stages starting with the first appearance of distinct cells in the primitive streak through to the early fetus. The isolated cells/tissues are incubated with the ligand display library. Methods of selecting ligands against cells isolated by microdissection of tissues have been described (Lu et al., Oral Surg. Oral Med. Oral Pathol. Oral Radiol. Endod. 98(6):692-7, 2004; Yao et al., Am. J. Pathol. 166(2):625-36, 2005). The isolated tissue/cells are washed to remove unbound phage and bound phages are recovered from lysates of the tissue using either infection of host bacteria or DNA amplification. When working with small amounts of tissue, DNA amplification is the preferred method of recovery because phage may lose infectivity when exposed to proteases present in tissue samples and because of the high sensitivity of DNA amplification (Burg et al., DNA Cell Biol. 23(7):457-62, 2004).

Screening of individual ligand display phage from the tissue selected pool is used to identify ligands that bind differentiation antigens. The selected ligand phages are screened against primate embryos at the appropriate stage of development using standard immunohistochemical methods to identify ligands that bind to the morphologically distinct cells from which they were selected. Ligands that bind to specific primate embryonic tissues are screened on human ES cells at various stages of differentiation to identify the equivalent human ES cells that have the human homologs of the primate surface antigens. In some cases cross reacting epitopes may identify human cells of other lineages which may also be of interest. It may take more than a single round of selection to reduce the complexity of the library sufficiently to allow screening of individual display phage. In this case, the selection is repeated with the enriched library incubated against an equivalent microdissected tissue sample until the library complexity is sufficiently reduced for screening of individual phage clones. It may also be advantageous to alternate selections of the library between a primate tissue sample at a particular stage of interest and a mixed population of stem cell suspected to contain progenitor cells of the same lineage to bias selection of the library toward phage particles that bind human progenitor cells of the same lineage as the primate tissue of interest.

### Example 3

### Identification of Ligands//Ligands that Bind Differentiation Antigens on ES Cells and Other Precursor Cells by cDNA Display

Description: In this example, the cDNA repertoire of ES cells or other precursor cells at various stages of differentiation is displayed on a suitable display particle (e.g. filamentous or T7 phage). The library of displayed proteins and protein fragments is used to immunize a mouse from which monoclonal Abs (MAbs) are selected using standard hybridoma technology. The resulting MAbs are screened for reactivity with the cells from which the library was prepared using standard ELISA assays.

Methods: Messenger RNA is isolated from ES cells or other progenitor cells and used as template for cDNA synthesis. For T7 phage libraries the cDNA library is prepared by standard methods and inserted into an appropriate T7 phage. For filamentous phage display, the cDNA is fragmented to display protein domains and ORF selected to increase the percentage of phage clones that display functional protein fragments (Zacchi et al., Genome Res. 13(5):980-90, 2003; Faix et al., Biotechniques 36(6):1018-22, 1024, 1026-9, 2004). Immunization with peptide display phage has been previously described and can be performed without adjuvant because the phage particles act as adjuvant. The resulting MAbs are screened against ES and other progenitor cells using cell based ELISA assays or other immunohistochemical screening techniques.

### Example 4

### Identification of Ligands/Ligands that Bind Differentiation Antigens on ES Cells and Other Precursor Cells Using Ligand Identification Via Expression (LIVE)

Description: The LIVE selection method has been previously described (Kassner et al., Biochem. Biophys. Res. Commun. 264(3):921-8, 1999; Legendre and Fastrez, Gene 290(1-2):203-15, 2002). ES or other progenitor cells are exposed to a phage library that displays a library of potential cell binding ligands/ligands and also contains a selectable or screenable marker gene such as neo or GFP. The cells are incubated to allow GFP expression from those phage that have bound and entered the cells (and therefore display cell binding ligands). Multiple rounds of selection and recovery of phage by DNA amplification are performed to reduce the complexity of the library. The ligand encoding phage DNA is then recovered by DNA amplification and sequenced to determine the sequence of the cell binding ligands. The GFP expressing cells may be incubated under conditions that allow further differentiation and phage DNA selected from differentiated cells. The ligand encoding phage DNA from such cells defines binding ligands that entered the cell at the time of phage incubation before differentiation occurred. In this way ligands that bind to progenitors of a defined lineage may be isolated.

Methods: ES cells or other progenitor cells are incubated with a phage display library containing a mammalian expression cassette with a screenable or selectable marker gene (Kassner et al., Biochem. Biophys. Res. Commun. 264(3):921-8, 1999) for 1 to 72 hours to allow phage internalization. The promoter for the screenable or selectable marker may be constitutive (CMV) or developmentally regulated.

When the promoter is developmentally regulated the cells are incubated under conditions that allow differentiation to occur and GFP cells are isolated and phage DNA encoding the cell binding ligands are recovered by DNA amplification. Multiple rounds of selection may be used to enrich for phage displaying cell binding ligands. Cell isolation is performed by FACs (for GFP), drug selection (neo gene), cloning cylinders or other methods known in the art. The cells may also be transplanted in-vivo and allowed to differentiate before isolation. Phage DNA amplification is performed by PCR or rolling circle amplification.

### Example 5

### Identification of Ligands that Bind Differentiation Antigens on Stem Cells using Combined Selection and Screening of Display Libraries

Description: Differentiation antigens that are characteristic of specific cell lineages are likely to occur very early in cultured stem cells. However, in the absence of an obvious change in the cell (such as the production of pigment in pigmented retinal epithelial cells) there is no way to distinguish which cells have differentiated along a specific pathway without specific markers of differentiation. In this example, cultured human ES cells are incubated under conditions that allow differentiation into one or more cell lineages for various periods of time (e.g. days to weeks). The selection strategy is to first select for any ligand display phage that bind the cultured stem cells and then screen individual phage or small pools of phage for phage displayed ligands that bind a specific subset of cells. Screening is performed in a multi-well plate format (e.g. 96 wells/plate) using standard immunohistochemical staining to detect bound phage. Positive pools of ligand display phages are deconvoluted to identify individual cell-binding phage.

Once ligands are identified that bind to a specific subpopulation of stem cells in a mixed population of cells, they are used to select additional ligands that bind the same specific cell population. A phage display library is contacted with a stem cell population as described above and incubated to allow phage binding. Unbound phages are removed by washing and the cells are dissociated from the plate into a single cell suspension. A specific subset of cells is isolated using affinity chromatography with the previously identified ligand on a solid support (e.g. magnetic beads). The bound phage are then recovered and amplified from lysates of the purified stem cell subpopulation using bacterial infection or DNA amplification. The process is repeated until the library complexity is sufficiently reduced to allow identification of additional ligand phage that bind the stem cell subpopulation.

Methods: A ligand display library is contacted to cultured human ES cells that are incubated under conditions that allow differentiation into one or more cell lineages for various periods of time (e.g. 2, 4, 6, 8 days) and unbound phage particles are washed off the cells using standard methods (e.g. low pH buffer). Bound ligand phage are recovered and amplified in host bacteria or by DNA amplification as previously described (Kassner et al., Biochem. Biophys. Res. Commun. 264(3):921-8, 1999; Burg et al, DNA Cell Biol. 23(7):457-62, 2004). The resulting display library is enriched for phages that display stem cell-binding ligands. The library is screened for specific cell-binding phage by plating at a density that allows picking individual phage clones which are then cultured in arrays (e.g. 96 well plates) with each well containing 1 to 20 individual library members. The arrayed phages are screened on cultured stem cells for binding to specific cell subpopulations within the total cell population. Following growth of host bacteria, the phage are rescued with helper phage and the plates are centrifuged to pellet the bacteria. The bacterial pellets are resuspended in growth medium and stored at 4° C. Bacterial supernatant is transferred to tissue culture plates containing human stem cell cultures and incubated to allow binding of ligand display phage. The stem cells are washed in phosphate buffered saline to remove unbound phage, then stained with anti-phage antibody, and visualized with a suitably labeled secondary antibody (e.g. phycoerythrin, fluorescein isothiocyanate) (Larocca et al., Mol. Ther. 3(4):476-84, 2001). Bound phage particles are visualized using fluorescence microscopy. Individual wells are examined for staining of cell-bound phage particles. Phage DNA from wells that score positive is recovered from bacteria grown on the original plate. The DNA is sequenced to determine the sequence of the binding ligand. For antibody libraries, the antibody gene is expressed in a suitable vector for further characterization of the antibody. The resulting ligands are further characterized by immunohistochemical staining to determine the pattern of expression of the target antigen on cultured human stem cells under various conditions and stages of differentiation and on embryoid bodies, teratomas, and non-human primate embryos, as well as tissues that contain stem cells such as fetal and adult tissues.

### Example 6

### Selection of Antibodies that Bind Differentiation Antigens on Stem Cells using Antibody Display Libraries Prepared from Non-Placental Animals

Description: Many of the stem cell antigens that one would like to raise antibodies against are likely to be recognized as self-antigens in the human and, therefore, might not evoke a sufficient antibody response in animals that are related to humans. This may include a large number of mammals because functionally important differentiation antigens would be expected to be highly conserved across species. Tolerization is a process where clonal expansion of B-cells that produce antibodies that bind self-antigens is suppressed to prevent autoimmune responses. It is advantageous when attempting to raise antibodies that bind human stem cell antigens to immunize a species that is sufficiently distant in evolution from humans such that it has not been tolerized to highly similar antigens. Chickens and humans are separated from a common ancestor by 310 million years of evolution, compared to about 87 million years of separation for humans and rodents. Chickens and humans share about 60 percent of their genes, as opposed to the approximately 88 percent shared by humans and rodents. Therefore, chickens and other evolutionarily distant animals are preferred over mice for raising anti-stem cell antibodies.

Monoclonal or polyclonal antibodies against stem cell antigens are prepared by immunization of chickens with whole live stem cells or extracts of cells at various stages of differentiation (e.g. 2, 4, 6, 8 days following incubation under conditions that promote differentiation). Monoclonal antibodies may be prepared using standard cell-fusion (hybridoma) techniques or by phage display. Phage display is preferred because it allows selection from a greater number of antibody clones (10 million to 1 billion) than cell-fusion (hundreds of clones).

In general, selection of antibodies from display libraries derived from immunized animals results in higher affinity antibodies than non-immunized animals because the antibodies that are initially selected undergo in-vivo affinity maturation as part of the natural immune response. However, it may be advantageous for making antibodies against certain highly conserved stem cell antigens to make a display library from naïve individuals as a means of by-passing immunization. Such libraries have been made from pooled human peripheral B-cells (Bradbury and Marks, J. Immunol. Methods 290(1-2):29-49, 2004) and are known to contain anti-self antibodies. In general, non-immune libraries result in lower affinity antibodies; however, the affinity may be improved by in-vitro affinity maturation using standard phage display mutagenesis and selection methods. Alternatively, synthetically derived antibody display libraries or combinations of synthetic and naïve libraries (Hoet et al., Nat. Biotechnol. 23(3):344-8, 2005) may be made using known methods from which anti-self antibodies can be selected.

Methods: Monoclonal or polyclonal antibodies against stem cell antigens are prepared by immunization of chickens with whole live stem cells or extracts of cells at that have been incubated under conditions that induce differentiation for various time periods (e.g. 2, 4, 6, 8 days). The chickens are boosted with additional injections of cells/lysates at, for example, 8, 15, 22, and 50 days following the initial immunization. Immunized animals are harvested at day 70, and mRNA is prepared from the spleen and used as template to synthesize cDNA using standard protocols (Sambrook and Russell, Molecular Cloning: A Laboratory Manual, 3rd Ed., Cold Spring Harbor Laboratory Press, 2001). The heavy and light chain variable region cDNAs are amplified from spleen using PCR and assembled into a single chain antibody using an appropriate linker and the appropriate synthetic oligonucleotide primers. The single chain antibody repertoire is ligated into a suitable phage display vector (e.g. the phagemid vector pCantab5) from which phagemid DNA is prepared and purified using standard protocols (Sambrook and Russell, Molecular Cloning: A Laboratory Manual, 3rd Ed., Cold Spring Harbor Laboratory Press, 2001). Alternatively the heavy and light chain variable regions may be expressed as Fab fragments by cloning into a suitable vector (e.g. pComb3). The phagemid DNA containing the antibody fragment repertoire is introduced into a suitable bacterial host (e.g. TG1) and rescued with a suitable helper virus (e.g. VCS-M13) to produce the phage particle library that is then purified from the culture medium using standard methods (Kay et al., Phage Display ofpeptides and Proteins: A laboratory manual, Academic Press, 1996).

### Example 7

### Identification of Peptides that Promote ES Cell Differentiation to Beta-Islets

Methods: A landscape phage library displaying 10 million different peptide sequences is attached to tissue culture plates in 10,000 pools of 1000 phage clones. Gene trapped hES cells are grown on the plates and screened for activation of a beta-islet cell lineage specific gene promoter (i.e. insulin) fused to GFP. Positive wells are re-screened and hits deconvoluted to identify differentiation associated peptides.

Library: Landscape phage display about 2700 copies of a peptide along the entire coat of the filamentous phage particle. A landscape filamentous phage display library of displaying 107 random 8mer peptide species is prepared using methods known in the art (Petrenko et al., Protein Eng. 9(9):797-801, 1996). In one embodiment the 8mer peptide contains an RGD or other known integrin binding sequence. The gene 3 protein on the landscape phage vector is modified by fusion of the gene to a peptide that binds the solid phase. For example, peptides may be used that bind plastic, or to other extracellular matrix components such as fibronectin. Alternatively, streptavidin or derivatives such as neutravidin are chemically conjugated to the tissue culture plate and a peptide sequence that is the substrate for biotinylation is fused to gene 3. Alternatively, the solid phase binding peptide is fused to the gene encoding the phage coat protein p9. Fusion to p9 leave the p3 protein intact so there is no interference with phage infectivity. The library is applied to the solid phase at a density of 10⁶ to 10⁹ phage particles/centimeter. The library is applied as a random mixture or in pools containing 1-1000 phage clones. The solid phase is a tissue culture plate, multi-well plate, or beads.

Selection and Amplification of Phage: Gene trapped ES cells are applied to phage coated plates and incubated under standard conditions. Conditions may be adjusted to allow cells to begin to differentiate. The cells are assessed for the presence of GFP expressing cells at regular intervals (i.e. daily). Cells that express GFP are lysed in wells or portions of cell culture (using cloning cylinders). When cells are grown on beads the beads with cells expressing GFP may be isolated by FACS or magnetic sorting. Selected phages are amplified in host bacteria by adding host bacteria directly to cell lysates in wells or cloning cylinders. Alternatively, selected phage DNA is amplified from phage using PCR or RCA amplification. Rescued phage are used to prepare an enriched library for subsequent selections by redistributing in multiwell format or for analysis of individual clones by DNA sequencing to derive the amino acid sequence of differentiation inducing peptides/proteins. An alternative to infection or amplification of phage DNA in-situ is to remove the phage from the plate or solid phase by enzymatic cleavage with, for example, subtilisin digestion or exposure to a protease when the cleavage site has been engineered into the phage coat protein that is used to bind the plate. An inducing peptide enriched library is prepared from the rescued phage or phage DNA using standard methods. The selection process is repeated for further enrichment such that the size of the pools is reduced to a small number of phages that are then screened as individual clones.

Assessment of Individual Peptides: Landscape phage bearing bioactive peptides that induce differentiation will serve as a renewable reagent that is used to grow cells along a defined differentiation pathway (i.e. insulin producing beta-islet cells). Peptides are produced using standard synthetic chemistry and chemically bound to tissue culture plates or extracellular matrix components. Larger differentiation inducing proteins are produced in bacteria, yeast or insect cells using standard recombinant DNA methods. Individual protein/peptides are tested for induction of differentiation using GFP gene trapped ES cell or normal ES cells that are tested for differentiation specific characteristics such as morphology, expression of unique cell surface receptors or other characteristics (i.e. glucose sensitive insulin secretion). Combinations of 2 or more individual selected peptides are tested in combination with various standard extracellular matrix components for affects on differentiation.

### Example 8

### Identification of Ligands at Multiple Time Points on Differentiating hESCs and the Developmental Lineage of hESCs Binding Such Ligands

Description: Methods of the invention are used to identify ligands that bind to cell surface receptors on stem cells at various stages of differentiation ranging from the start of differentiation up to 8 weeks or longer. Differentiating ES cells are contacted at various time points with a library of genetic packages displaying potential ligands (i.e. a phage display library) that recognize and bind to cell surface receptors. After incubation with the cells, the unbound peptide display phages are removed and the bound phages recovered from the cells. Following enrichment of the library for cell-binding phage, the selected peptide phage clones are tested individually to determine the specificity of binding to differentiated cells at various stages of development and the developmental fate of the cells that internalize peptide phage is mapped.

Procedure: Selection of Cell-Binding Ligands: Embryonic stem cells are cultured by methods known in the art. Klimanskaya et al., Cloning Stem Cells 6:217-45, 2004; Thomson et al., Science 282:1145-7, 1998. On day 0, the cells are cultured under conditions that are permissive for differentiation. Differentiation is accomplished by various methods known in the art including chemical induction, co-culture with inducing cells or tissues, and spontaneous differentiation. Odorico et al., Stem Cells 19:193-204, 2001; Schuldiner et al., Methods Enzymol. 365:446-61, 2003; Schuldiner et al., Proc. Natl. Acad. Sci. USA 97:11307-12, 2000. In this example differentiation is induced by allowing the cells to attain extensive cell-cell contacts (confluency and overgrowth of cells on the culture support). A ligand display library is contacted with the cells at day 2, 3, 4, 5, 6, 7 10, 12, 14, 16 and every 2 days thereafter up to 8 weeks in culture. In this example, the ligand display library is a cysteine constrained random 7 amino acid sequence (CX7C) that is displayed in the T7 Select phage vector (Novagen).

The peptide display library is contacted with the cells for 4 hours at 4° C., followed by repeated washing of the cells on the plate with PBS to remove unbound phage (at least 6×). The washed cells are removed from the plate using an EDTA solution in PBS and washed an additional 3-5× in PBS containing 1% BSA. The cells are lysed in PBS containing 1% NP40, contacted with bacterial host cells (i.e. E. coli strain BL21), and amplified by growth in the host bacteria. The resulting phage population is enriched for peptide display phages that bind to differentiating ESCs. The peptide sequences are determined by sequencing the DNA from individual phage clones containing the peptide encoding sequence. The enriched library is further enriched by repeating the selection procedure. A 10 fold increase in retention of phage library by the cells compared to retention of a control phage (no displayed peptide) is indicative of successful selection of specific cell binding peptide phage. Increased retention of phage as measured by the percentage of input that is cell-bound occurs in as little as 2 rounds of selection. Typically the displayed peptide library is reduced in complexity from 10 of millions of random peptides to between 1 and several dozens peptides or peptide families within 3-5 rounds of selection. Alternative methods for recovering phage are known to result in recovery of specific phage in as little a 1 or 2 rounds of selection. Burg et al., DNA and Cell Biology 23:457-462, 2004.

Ligands are selected that bind to one or more cell types present in the culture at the time of phage-cell culture contact. Certain ligands bind surface receptors that are unique to a particular progenitor/precursor cell type while others recognize receptors that are common to 2 or more types of progenitors. Based on what is known about hematopoietic cell differentiation, it is reasonable to assume that a unique ligand or combination of ligands will be specific for each type of progenitor in the culture. By surveying the differentiating ESCs with ligand display libraries at multiple time points during differentiation up to 8 weeks (end of embryonic development) we obtain ligands that bind to all progenitor and precursor cell types at various stages in their differentiation into the multitude of cell types in the developed embryo/early fetus. A list of the differentiated cell types in the adult human may be found, for example, at the Wikipedia web site entitled “List of distinct cell types in the adult human body”.

Determining Specificity of Selected Peptide Ligands: The peptide ligand display phage is incubated with differentiating hES cells at various stages of differentiation from 1 day to 8 weeks and the cells are stained using a fluorescently labeled secondary Ab under both permeabilized and non-permeabilized conditions to assess internalization and surface binding. Alternatively the peptide is made synthetically and conjugated to a fluorescent tag. The tagged peptide is contacted with differentiating hESCs to assess which cells bind peptide. Cell staining with peptide or peptide-phage is performed at various time points during differentiation (from 0-8 weeks). For quantitative measurement of peptide binding the phage bound-cells are removed from the plate, exposed to labeled secondary Ab and sorted by FACS to determine the percentage of cells that bind ligand.

Tracking the Developmental Lineage of hESCs that Bind Peptides: One method of tracing the lineage of cell that internalize specific peptides is to use the peptide to specifically introduce a stable genetic change in the recipient cell via receptor-mediated DNA delivery. In this method, the peptides are used to direct a GFP expressing viral vector (typically adenovirus) into the receptor bearing cells. The adenoviral vector approach can be used with internalizing ligands or ligands that do not internalize since internalization is carried out by integrin binding sequence in the viral knob coat protein 7. Wickham et al., Cell 73:309-19, 1993. One way this approach is applied to confer cell-binding peptide tropism to a GFP adenoviral vector using the adenobody strategy. Watkins et al., Gene Ther. 4:1004-1012, 1997. Adenobodies are created by engineering an anti-knob scFV to display the cell-binding peptide as a peptide fusion on the end opposite of the Ab binding fragment. Retargeted adenovirus having the appropriate specificity is used to introduce GFP expression in the peptide targeted cells and the fate of GFP expressing cells is tracked using fluorescent microscopy.

An alternative to peptide directed adenoviral gene delivery is to use the peptide display phage themselves to deliver a reporter gene. Previous studies have shown that receptor-mediated internalization of ligand-phage is highly specific for cells that bear the cognate receptor using a variety of ligand/receptor pairs. Larocca et al., Human Gene Therapy 9:2393-2399, 1998; Poul et al., J. Mol. Biol. 288:203-211, 1999; Kassner et al., Biochem Biophys Res Commun. 264:921-928, 1999; Larocca et al., Curr. Pharm. Biotechnol., 3:45-57, 2002. Other methods include conjugating the peptide to a DNA condensing agent such as poly-L-lysine and using the peptide polylysine condensed DNA to specifically introduce reporter gene DNA into the peptide targeted cells. Hart et al., Gene Ther. 2:552-4, 1995. Non-genetic methods of tagging cells with internalizing peptide phage or peptides may also be used. Peptide phage may also be labeled with a long-lived radio-isotope (e.g., ³⁵S), with Quantum Dots, or with another suitable label.

### Example 9

### Selection of Peptide Display Library on Mixed Progenitor Cell Population

Peptide phage selection studies were performed using an mESC line containing a myosin heavy chain α (MHCα) promoter regulated eGFP gene. The MHC promoter extends from −5446 bp to −4 bp relative to ATG and includes non-coding exons 1,2 and UTR of exon 3. Similar approaches may also be used with the NIH-approved hESC H9 line (NIH registry WAO9) containing the equivalent MHC α-eGFP expression cassette resulting in fluorescence in cardiomyocyte populations, and with other ESC cell lines that have been appropriately engineered.

A selection experiment was performed to determine conditions and feasibility of selecting a phage library against differentiating mESCs. mESCs were used because their low cost, and relatively short doubling time compared to hESCs facilitates the optimization of conditions for phage selection recovery. The mESC line, CGR8-MHC-eGFP, was chosen because it was engineered to express an eGFP gene regulated by the cardiomyocyte specific MHCα promoter. The cardiomyocyte-specific eGFP expression was used to assess recovery of phage DNA by PCR after isolation of cardiomyocytes by FACS.

A cysteine-constrained 7mer random peptide library (CX₇C) was generated in the T7 select-415 vector (Novagen). The CGR8-MHC-eGFP mouse embryonic stem cells were plated onto non-adherent Petri dishes in media lacking LIF1 and grown for 4 days under standard tissue culture conditions to allow the formation of embryoid bodies. The 4 day-old embryoid bodies were plated on 100 mm tissue culture dishes that were coated with 0.1% gelatin and incubated for 36 hours prior to phage addition. The phage library (2×10¹⁰ pfu) was added to cells and incubated for 4 h at 4° C. Cells were washed in PBS (6×10 ml) at room temperature, removed from the plate with 0.536 mM EDTA solution and washed 3×14 ml with PBS containing 1% BSA. The washed cells were lysed in PBS/1% NP40 and phage recovered by infection of BL21 host bacteria as previously described. Laakkonen et al., Nat. Med. 8(7):751-755, 2002. Recovered phage were titered to determine output and amplified to generate input phage for the subsequent selection round.

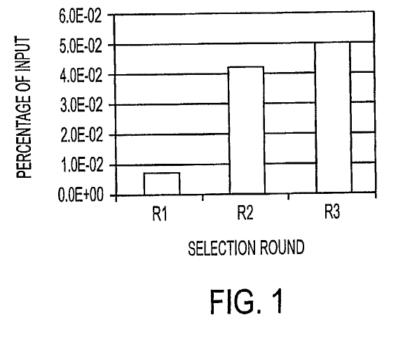

The results of 3 selection rounds are shown in FIG. 1. A 7-mer constrained peptide display library was selected against differentiating mESCs (˜3 million cells). The cell bound fraction of the library was used for each subsequent round of selection. Percentage of input=phage added (pfu)/phage recovered (pfu)×100. An increase in the output ratio (output/input) indicates successful enrichment of the library for phage displayed peptides that bind cells in the differentiating mESC population.

### CRIP2-Binding Peptide Selected by Phase Display

Sequencing of a sample of 42 phage clones from the round 3 enriched library revealed that families of related peptides were selected (Table 1). A high percentage of the sampled sequences share a common RXXR motif, and most of these contain a C-terminal arginine residue. The protein prohormone and growth factor processing enzyme, Furin, has been shown to have substrate specificity for the RXXR motif. Matthews et al., Protein Sci. 3(8):1197-1205, 1994. The peptides that contain the C-terminal arginine may therefore mimic one or more peptide hormones that are normally processed by Furin or similar enzymes. Furin is highly expressed in the primitive heart (8.5 dpc), and the number of myocardial precursors is diminished in far −/− mice, which are defective in embryonic turning and heart looping. Constam and Robertson, Development 127(2):245-54, 2000. Although the enriched library was not specifically selected for cardiomyocyte progenitor binding peptides, the data indicate that a high percentage of the sequences identified in the round 3 library are possible heart targeting peptides.

TABLE 1
Cell-Associated Peptide Sequence Families.

| | | |
|---|---|---|
| CRPPR* (2) | CTLPRLKRC | CSLVSSKRC |
| CRPAR* | CANRPPR* | CRVSSKDKC |
| CRTKR* | CEMRPPR* | other unique seq (24) |
| CRAPR* | CVKRPLR* | |
| CRGPR* | CLRPRRGDC | |
| CRSPR* | CQRNGRNPC | |
| CRSVRGGAKLAAALE* | CSRAPRTKC | |
| | CSRSAR* | |

Derived amino acid sequences of phage displayed peptides after 3 rounds of selection on differentiating mESCs (d5.5). Of 42 phage clones sequenced, 16 contained the RXXR motif and 2 contained VSSK.

### Example 10

### Selection of Ligands that Bind Specific Progenitor Cells Over Time

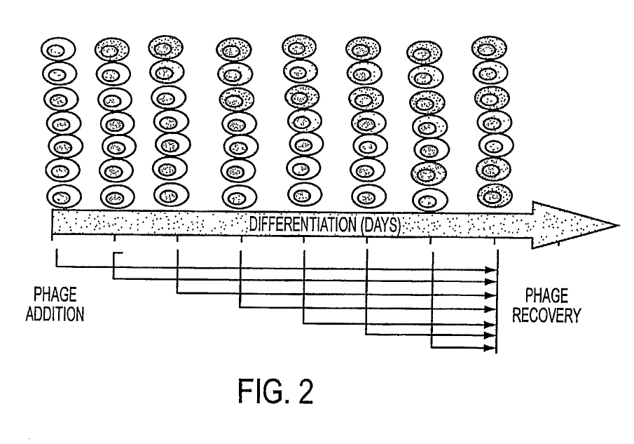

Description: Methods of the invention are used to identify ligands that bind to cells that change over time. The methods may be used to find ligands that identify the progenitor of a cell that has changed over time regardless of whether the receptor for the ligand is remains expressed on the cell surface. For example, they may be used to find the precursors of cells that have differentiated over time into specialized cell types. When selecting peptide display libraries on a mixture of differentiating ESCs, it is likely that the selected peptides will bind to many different receptors present on a variety of cell types in the culture. To select ligand display phage that bind to a specific progenitor cell type it is advantageous to have a means of isolating the specific population of interest. Currently there are very few well defined molecular markers for differentiating ESCs. They are almost always intracellular and define progenitors of many different cell types. In contrast to early precursor cells, differentiated cells are often easy to distinguish morphologically and molecularly and thus, it is advantageous in this regard to allow further time for isolation of these cells and the specific peptide phages that bind to them. This approach differs from standard phage display in that it incorporates an extended “time lapse” between the addition of a phage display library and the recovery of phage from the cells of interest. In the example of differentiating cells, the cells of interest are separated from surrounding cells using an antibody against a marker of the differentiated cell and fluorescent or magnetic based sorting (FACS/MACS) or using cells that are engineered to express a cell-specific reporter gene that is the basis for FACS/MACS. Multiple selections may be performed at regular intervals on differentiating cells to obtain multiple “molecular pictures” of the target cells at regular intervals over time much the way “time-lapse” photography allows the viewer to see changes in a subject that take place over time (FIG. 2).

Procedure: The method consists of contacting the cells with a peptide display library, removal of unbound phages, incubation of cells for time period allowing differentiation to occur, isolation or enrichment of differentiated cells of interest and recovery of peptide phage from cells of interest.

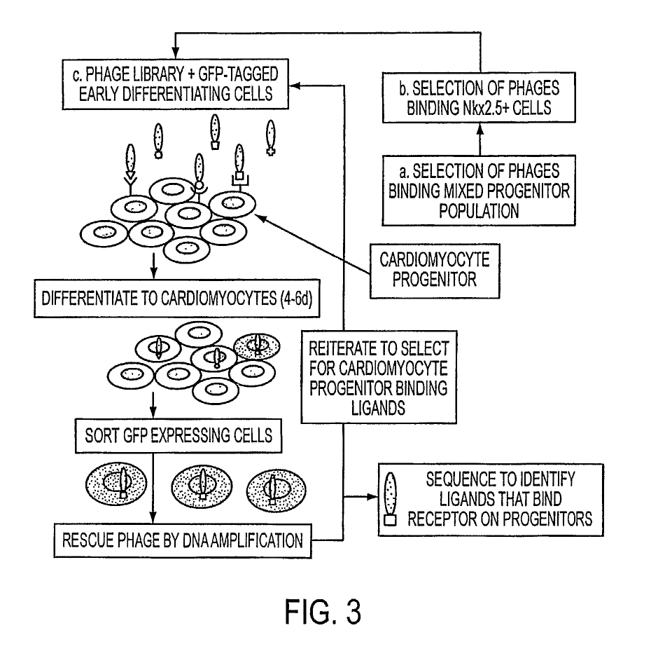

An example of the method as applied to identification of peptides that target progenitors of cardiomyocytes is shown in FIG. 3. The peptide display library is selected against differentiating ESCs (1-2 days after plating embryoid bodies) in a series of increasingly stringent selection strategies starting with (a.) selection for binding to all differentiating cells (b.) selection for binding to NRx2.5+ cells using FACS and (c.) selection for phage that internalize into cardiomyocyte progenitors using time-lapse phage display (TLPD). Each selection is reiterated until the library has at least 10-fold greater retention by cells than insertless control phage. For selection with TLPD, unbound phage are removed by washing and the cells are allowed to incubate an additional 4-7 days to allow differentiation to occur. Phage that have internalized into the progenitors of cardiomyocytes are recovered from GFP positive cells by PCR.

Selection of Ligands that Bind Progenitors of Cardiomyocytes

Contacting differentiating mESCs with phage library: Mouse embryonic stem cells that have been engineered to express GFP under the control of a cardiomyocyte specific transcriptional promoter are placed under conditions that promote the formation of embryoid bodies (day 0). On day 4 the embryoid bodies are allowed to attach to gelatin coated tissue culture plates. On day 5-6 the ligand display library is incubated with the cells for 4 hours at 4° C. and unbound phage are removed by washing 6× with PBS. The ligand display library is a cysteine constrained random 7 amino acid sequence (CX₇C) that is displayed in the T7 Select phage vector (Novagen). Fresh media is added and the cells are returned to the incubator at 37° C. to allow phage internalization. The phage treated cells are cultured for an additional 8 days to allow differentiation of progenitors into cardiomyocytes at which time the cells are harvested by dissociation with collagenase to obtain a single cell suspension. The cardiomyocytes, which express eGFP, are isolated by FACS and the peptide encoding phage DNA is recovered by PCR from total cell DNA. The PCR product is purified and digested with restriction enzymes appropriate for insertion into the T7 select vector arms. The selected peptide phages are prepared by ligation of the enzyme cut DNA with restriction enzyme digested T7 vector arms and in-vitro packaging (Novagen).

The derived amino acid sequences of a representative sample of phage clones obtained from amplification of DNA extracted from EGFP expressing cells is shown in Table 2. Phage clones displaying peptides that contain the consensus sequence K/RXXR or K/RK/RXXR are highly represented indicating selection for these sequences. Many of the consensus containing sequences share a core consensus sequence or are identical with peptide phage sequences that are selected using standard phage selection on mESCs but only after 3-5 rounds of standard selection (Table 3). These data indicate that TLPD is more sensitive than traditional phage display for selecting specific binding sequences from large libraries of random peptides.

TABLE 2
Sequences of selected peptides from round 1 of TLPD on mESCs.

| | K/RXXR | K/RK/RXXR | |
|---|---|---|---|
| CKNGTRAKC | CRAKPPR* (3) | CRKAPR* (4) | CLLD* |
| CRSAGGDPC | CGRPPR* (2) | CRKPVR* (2) | CDP* |
| CPKTGSDLC | CRPPR* | CPKRPVR* | CG* |
| CRNSVKSPC | CKPIR* | | |
| | CNKPQR* | | |
| | CGGKPAR* | | |
| | CNKTNRKGC | | |

Sequences containing the K/RXXR consensus are highly represented (17/24).
Sequences containing the K/RK/RXXR are also highly represented (7/24).

TABLE 3
Similarities between sequences from round 1 TLPD
and sequences from round 3-5 of standard selection
of the same peptide display library on mESCs.

| Round 1 | Round 3 | Round 4 | Round 5 |
|---|---|---|---|
| TLPD | Standard | Standard | Standard |
| (n = 24) | (n = 47) | (n = 12) | (n = 12) |
| CKPIR* | CALKPIR* | | |
| CRPPR* | CRPPR* (2) | CRPPR* (3) | CRPPR* |
| CGRPPR* (3) | CEMRPPR* | CKPPR* | CRAKPPR* |
| CRAKPPR* (2) | CANRPPR* | | |
| CRKAPR* (4) | CRAPR* | | CRKAPR* |
| CPKRPVR* (2) | | CPKRPVR* | |
| CRKPVR* | | | |

Most of the round 1 TLPD selected sequences containing R/KXXR were also identified using standard selection but only after 3-5 rounds of selection.

The binding of individual peptide display phage to mESCs is tested by adding phage to mESCs and recovering cell-associated phage using the same method used for selection of the library. The ratio of phage output/phage input is compared with that of a control T7Select phage (which does not display a peptide) (Table 4). Sequences that contain a C-terminal arginine are the strongest cell binders; in contrast, cysteine constrained sequences with RXXR consensus sequence do not bind significantly more than control phage (<10-fold). For example, CSRAPRTKC contains a similar core consensus sequence as CRKAPR* but binds much less efficiently as the sequence with a C-terminal arginine. In a single round of selection, TLPD identifies peptides with strong binding to differentiating ESCs relative to un-targeted control phage.

TABLE 4
Binding of individual peptide display phage
clones compared to control phage.

| Peptide display phage clones | Peptide display phage clones |
|---|---|
| that bind mESCs >100-fold | that bind mESCs >10-fold |
| over control phage. | over control phage. |
| CRSPR* (R3 standard) | CTLPRLKRC (R3 standard) |
| CRPPR* (R1 TLPD) | CGVQRQPKC (R3 standard) |
| CPKRPVR* (R1 TLPD) | CLGPRKKAC (R3 standard) |
| CRKAPR* (R1 TLPD) | CSRAPRTKC (R3 standard) |

Binding to mESCs is strongest for clones that display peptides that contain a C-terminal arginine. Sequences with the RXXR consensus but lacking the C-terminal arginine have low binding retention on mESCs.

### Example 11

### In-Vivo Time-Lapse Phase Display

The methods of the invention are amenable to both in-vitro selection as described above as well as in-vivo selection in experimental animals. For example, a random peptide phage display library is injected into the embryo of an experimental animal such as a chicken or mouse and cells of various differentiated tissues are isolated from the animal after allowing sufficient time for development and differentiation to occur. The peptide phages that are internalized by the progenitors of the specific tissues are identified by extracting DNA from the tissue and amplifying the phage DNA encoding the peptide ligand(s) displayed by the phage using PCR or other suitable DNA amplification methods. The ligand encoding sequence is ligated into the phage vector to amplify phage for sequence analysis and/or additional cycles of selection. This method is particularly useful for identifying progenitors of cells or tissues for which the precise time location of the progenitors in the embryo is unclear. For example, to identify the hemangioblast cell that is the precursor of both the endothelial and hematopoietic cells, a phage library is injected into the AGM region of an embryo, the embryo is allowed to develop into a fetus and phages that internalized in progenitor cells are recovered from the early blood cells in the fetal liver. A similar approach is applied to find cell surface markers that identify precursor cells of the beta islet cells within the developed pancreas. Another variation of the method is to add a phage library to early differentiating ESCs in-vitro, implant the cells in-vivo to allow formation of a teratoma and recover phage from specific differentiated cell types. Whether identified using in-vitro ESCs or in-vivo, peptides that bind early precursor of differentiated tissues are useful for identifying the presence of such precursors in both embryonic and adult tissues.

### Example 12

### Sequences Identified by Selection of a Peptide Display Library on Human ESCs Include Convertase Substrate Motifs

A CX7C (cyclized random 7-mer peptide) library is selected against human ESCs (H9 cell line, WA09) that are cultured under differentiation conditions for 6 days. Klimanskaya et al., Cloning Stem Cells 6:217-245, 2004. The cells are grown on mitomycin C treated mouse embryonic fibroblasts (MEFs) and for differentiation are allowed to overgrow on MEFs with the culture medium replaced daily for 6 days. The library is selected using 2 selection strategies. In one strategy (N), the library is selected directly as is and in the other (P), the library is incubated sequentially with MEFs, followed by undifferentiated hESCs to pre-adsorb peptide phage that bind these cells prior to selection on differentiated embryonic cells.

The retention of phage by the cells is found to increase about 10-fold after each selection, indicating that the library is enriched for cell-binding phage. The percentage of input phage that is retained by the cells at round 3 is about 0.1% indicating relatively strong cell binding. Twenty four peptide phage clones are sequenced for each strategy from the 3^{rd} round of selection (Table 5). There is a high percentage of (K/R)XX(K/R) motif containing sequences. The P strategy resulted in twice as many (K/R)XX(K/R) sequence clones as the N strategy. In addition to the (K/R)XX(K/R) motif, 2 other motifs are prevalent ((K/R)(K/R)(K/R) and (K/R)X(K/R)X(K/R)). All 3 motifs were also found in the 3^{rd} round of mESC selected phage, however, the (K/R)XX(K/R) motif is most abundant. The KRTS motif is present only in the human round 3 from the N selection. The KRTS sequence is found in embryonic cell specific gene 1 (Genbank XP292301) and in retinal pigmented epithelial spondin-like protein (Genbank XP497769).

TABLE 5
Sequence families in representative sample of hESC selected
peptide display phage after 3 rounds of selection.

| (K/R)XX(K/R) P | (K/R)XX(K/R) N | ((K/R) (K/R) (K/R) P | ((K/R) (K/R) (K/R) N |
|---|---|---|---|
| CRRVPR* | CRKQPR* | CGRRK* | CRRR* |
| CRRTPR* | CQKRVR* | CASRRK* | CLQRKRGTC |
| CRRAPR* | CNKKSRGSC | CMIKRKATC | CNIARRKAC |
| CRKPR* | CHKKGKS* | CQGRKRLAC | CRDLGKRKC |
| CGSRKPR* | REVKRAKC | CQGVRKKVC | |
| CSRKPR* | CKGKTARSC | CAAPPRRKC | |
| CSRRAKLVC | CFTRANRKC | | |
| CTRRAKASC | | | |
| CGLKRAKSC | | | |
| CGGSRKSKC | | | |
| CRSLKRGSC | | | |

| (K/R)X(K/R)X(K/R) P | (K/R)X(K/R)X(K/R) N | KRTS P | KRTS N |
|---|---|---|---|
| CRPKSRVGC | CKMRART* | NONE | CKRTSARQC |
| CQKSRARMC | CKTKGKSAC | | CAKRTSKAC |
| | CKPRTKSLC | | |
| | CKGRNKGIC | | |

TABLE 6
Frequency of sequence families in human preadsorbed
library, non-preadsorbed library and in the
round 3 phage selected on mESCs.

| Motif | Human R3 (N) | Human R3 (P) | Mouse R3 |
|---|---|---|---|
| (K/R)XX(K/R) | 29% (7/24) | 58% (14/24) | 43% (20/47) |
| (K/R) (K/R) (K/R) | 17% (4/24) | 25% (6/24) | 4% (2/47) |
| (K/R)X(K/R)X(K/R) | 17% (4/24) | 8% (2/24) | 2% (1/47) |
| KRTS | 8% (2/24) | 0% | 0% |

Similar sequences are obtained for human and mouse and in some cases the core consensus sequences are identical (Table 6). These data indicate that the receptors for the selected peptides are highly conserved from mouse to human. Indeed, a mouse selected sequence CRPPR* binds and internalizes into a subpopulation of hESCs. The CRPPR sequence is known to bind to adult heart capillaries and endocardium. Zhang et al., Circulation 112:1601-1611, 2005. It binds to CRIP2 (also known as heart LIM protein (Hlp), which is expressed in the heart-forming primordia and the developing heart. Zhang et al., Circulation 112:1601-1611, 2005; Yu et al., Mech. Dev., 116(1-2):187-92, 2002. The (K/R)XX(K/R) motif fits the minimal recognition site for the subtilisin-like furin family of prohormone processing proteases. Matthews, D. J., et al., Protein Sci. 3(8):1197-205, 1994. It is likely that convertases play an important role in development. For example a furin negative mouse is defective in embryonic turning and heart looping and has diminished numbers of myocardial precursors. Roebroek et al., Development 125(24):4863-76, 1998; Constam and Robertson, Development 127(2):245-54, 2000. The products of furin or similar convertase enzyme cleavage have a C-terminal arginine and thus the peptides may mimic the processed hormone. Such processed hormones include members of the FGF and TGFbeta protein hormones which are known to be active during differentiation. The strongest peptide binders from the mouse selection are those that contain a (K/R)XX(K/R) motif and a terminal arginine. There are 6 such sequences in the P selected sequences and 2 in the N selected. The increase in the (K/R)XX(K/R) motif containing peptides (particularly those with terminal arginines) in the preadsorbed library indicates that the preadsorbsion helped enrich for peptides that bind differentiated cells. The binding of the selected convertase substrate-like peptide sequences indicates that differentiation-specific convertases can serve as markers of differentiation in embryonic cells. Alternatively, the cell surface markers of differentiation are receptors for convertase processed protein hormones that share homology with the selected peptide display phage.

### Example 13

### Time-Lapse Phase Display

### Fate of Internalized Phase DNA

Description: Recovery of phage DNA after a prolonged period of time allows the phage treated cells to differentiate into cells expressing cell-specific markers that are characteristic of a particular differentiated cell-type. In certain cases it is advantageous to recover the phage DNA from cells that have been cultured for several weeks or more. We tested the feasibility of recovering internalized phage DNA by incubating a known internalizing phage with differentiating embryonic cells. We assessed whether internalized phage DNA was recoverable from cells that were incubated for as long as 18 days after exposing the cells to phage.

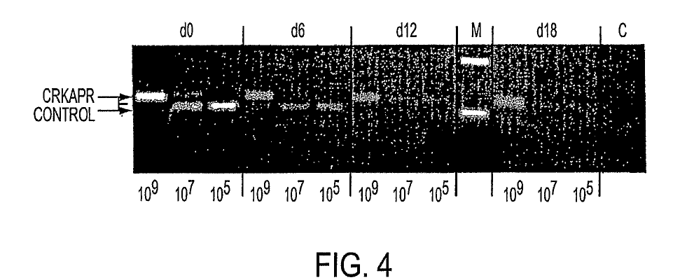

As shown in FIG. 4, a mixture of CRKAPR-phage and control phage was incubated with mouse differentiating embryonic cells (d5.5) in 6 well tissue culture plates (about 1-3×10⁶ cells/well). The CRKAPR phage was added at 10⁹, 10⁷, or 10⁵ pfu/well and mixed with 10⁹ pfu/well of control phage in PBS+10% FBS at 2 ml/well. The phage mixtures were incubated with the cells for 6 hours at 37° C. followed by 6×3 ml washes with PBS. Cells were then either harvested directly or returned to the incubator in differentiation medium. Cells were harvested for DNA extraction at 0, 6, 12, and 18 days after phage incubation. Phage DNA was amplified from 100 ng of total cellular DNA using primers that flanked the peptide encoding DNA insert using nested PCR.

Procedure and results: The survival of internalizing phage displaying the CRKAPR peptide was compared to control phage with no foreign peptide displayed after incubation with target cells. The 2 phage were added together at ratios of peptide:control phage of 1:1, 1:100, and 1:10,000. The DNA was extracted from the cells by proteinase K treatment followed by phenol extraction and precipitation with ethanol. Sambrook and Russell, Molecular Cloning: A Laboratory Manual, 3rd Ed., Cold Spring Harbor Laboratory Press, 2001. Phage DNA encoding the peptide or insertion site for the control phage was amplified using nested PCR (20 cycles with outside primers; 25 cycles with internal primers). When equal amounts of CRKAPR and control phage were added, the CRKAPR phage DNA (upper band) was easily detected with little or no control phage DNA amplification at all time points up to 18 days. When 100 or 10,000-fold excess control phage was added, the control phage was the predominately amplified product at day 0 and day 6. However, only CRKAPR phage DNA was the detectable amplified DNA product at day 12 and 18. These data indicate that the control phage DNA is degraded more rapidly than the CRKAPR phage DNA and that the fraction of the internalized CRKAPR phage DNA that survives can be detected by PCR as long as 18 days after internalization. In the presence of excess control phage, the CRKAPR phage DNA was low or undetectable by PCR at 0 and 6 days because it is out competed for PCR priming and subsequent amplification by the control phage DNA.

### Example 14

### Tracking Quantum-Dot Labeled Internalizing Phage During Differentiation of Embryonic Cells

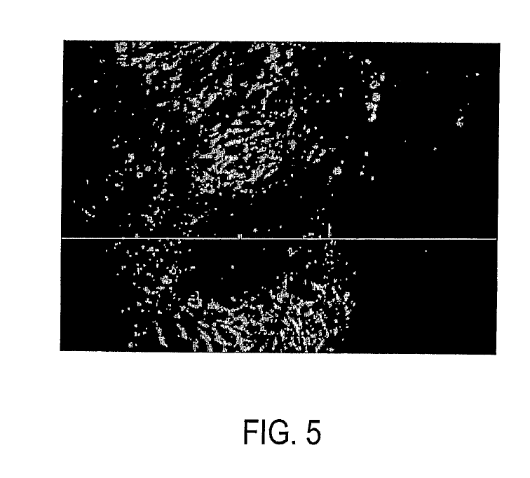

Selected phage and/or synthetic peptides derived from the displayed sequences are screened to determine the developmental fate of cells that express the receptor for the selected peptides. For these purposes, it is important that the signal from the quantum dots is stable. In this experiment, we labeled mouse differentiating embryonic cells (DECs) with CRKAPR peptide phage that were conjugated to quantum dots (655 nm) through an avidin-biotin bridge. The phage particles are purified using PEG precipitation and biotinylated using Sulfo-NHS-Biotin LC (Pierce). A final PEG precipitation is used to remove free biotin. The phage are added to streptavidin conjugated to 655 nm quantum dots (Invitrogen). In this example the CRKAPR peptide phage, which binds strongly to mouse DECs (400-800 fold over control phage) was tested using quantum dot labeling and visualization in targeted cells. As shown in FIG. 5, biotinylated CRKAPR-display phage were added to differentiating embryonic cells (CGR8-MHCα at d 5.5) grown on plastic 8 well chamber slides for 16 h at 37° C. Free phage particles were washed away and the cells cultured for an additional 30 days. Internalized quantum dots are visible as small bright spots. Differentiated cardiomyocytes expressing GFP regulated by the myosin heavy chain α promoter are visible by green fluorescence throughout the cytoplasm of the elongated cardiomyocytes.

The quantum dot signal was relatively strong in peptide phage treated cells compared to quantum dot labeled control (insertless) phage. The signal was observable at 16 h, 4 days, 24 days, and 31 days after phage addition. The minor loss of signal observed is probably the result of dilution as the cells replicated. The cells targeted by this peptide appear to follow a developmental fate that is largely distinct from cells that differentiated into cardiomyocyte as shown by the limited overlap between the two cell populations. However, confocal microscopy revealed a small population of cells that were both GFP and q-dot positive (not shown).

### Example 15

### Identification of Ligands that Bind Dermal Fibroblast Progenitors

Early dermal fibroblast like cells can be differentiated from hESCs by selection of clonal populations from ESCs grown at low density in differentiation inducing medium. However, the proliferative capacity of certain clonal populations may be limited, thus, limiting their analysis. Therefore, it would be advantageous to select peptides that bind surface antigens on early hDECs that will be used to isolate the precursors of dermal fibroblasts. Isolation of surface marker tagged cells from early differentiating hESCs would provide a readily renewable source of cells for making in-vitro cultured skin equivalents.

A CX7C cyclic peptide display library is contacted with early differentiating hESCs (day 6), washed to remove non-binding phage and incubated an additional 16 hours to allow internalization. Non-bound phage are washed away and the phage treated cells are removed from the plate and replated on 15 cm gelatinized plates and grown for an additional 14 days for further differentiation. The cells are then replated at low density on gelatinized 15 cm plates and colonies isolated after 7-10 days. The cells from each clone are are harvested and split, with one half for PCR and phage DNA recovery and the other half for expansion and characterization using gene chip analysis. Phage DNA encoding the ligand that resulted in internalization into the dermal fibroblast progenitors are recovered from the differentiated clones by nested PCR amplification and the EcoR1/HindIII inserts cloned back into the T7-select vector to reconstitute the recovered phage for the next round of selection and DNA sequence analysis. The selected peptide display clones are characterized using lineage tracking with quantum dot labeled phage. Individual peptide phage clones are screened for those that can introduce the quantum dot label into early cells that become fibroblast like cells upon prolonged incubation on gelatinized plates. Selected peptides that meet this criteria are used to isolate precursor cells from cultures of early differentiated hESCs. The isolated cells are analyzed by Illumina genome expression analysis for markers of early dermal fibroblasts. Clonal cell lines are derived as described, e.g., in U.S. Patent Application Nos. 60/738,912, filed Nov. 21, 2005, 60/791,400, filed Apr. 11, 2006, and 60/798,103, filed May 4, 2006, the disclosures of which are incorporated herein in their entireties. Candidate cells are introduced and assessed as the dermal component organogenic skin cultures.

### Example 16

### Regulation of the Expression of Prohormone Convertases in Differentiated hESCs

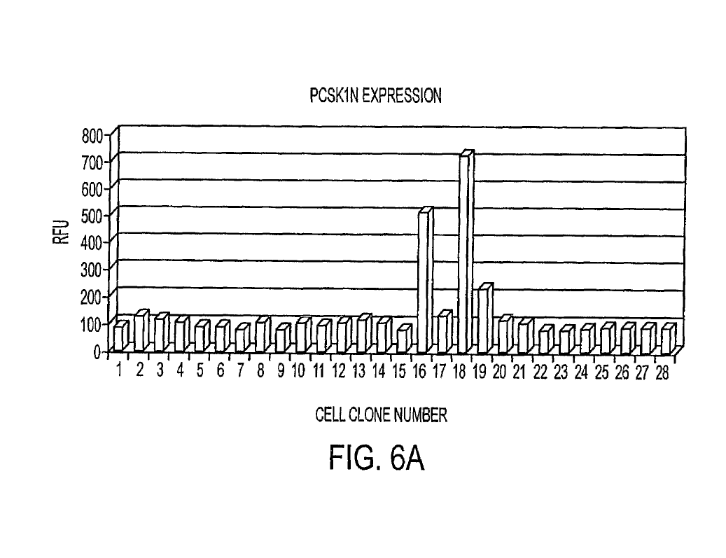

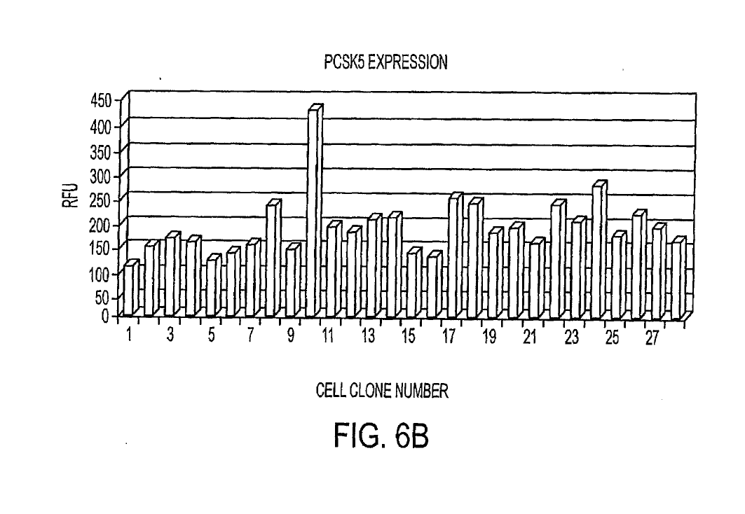

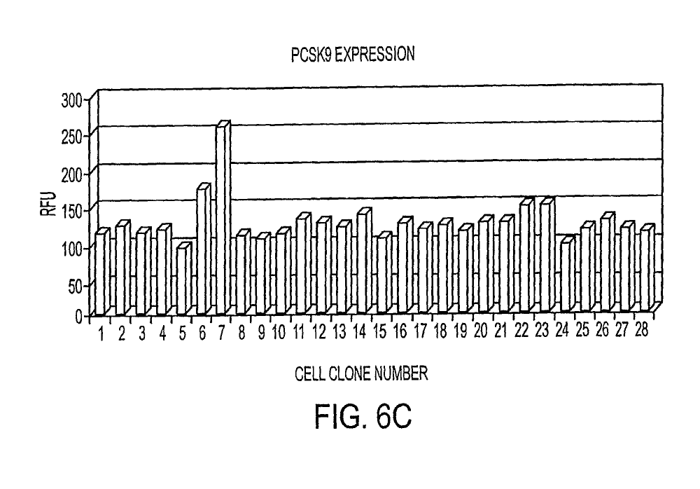

Certain clonal lines of cells derived from differentiated hESCs overexpress either prohormone convertases (PCSK9, PCSK5) or an inhibitor of the prohormone convertase PC1 (PCSK1N) (see FIG. 6). Without wishing to be bound by theory, taken together with the discovery of RXXR containing peptide ligands that bind early differentiating hESCs, the data are consistent with the idea that expression of certain processing enzymes may play an important role during development by activating or inhibiting peptide hormones or growth factors that stimulate or inhibit differentiation. Thus, the peptides that are selected using phage display methods described here may be used to regulate the differentiation of stem and other progenitor cells by modulating the activities of proprotein convertase enzymes. Clonal cell lines are derived as described, e.g., in U.S. Patent Application Nos. 60/738,912, filed Nov. 21, 2005, 60/791,400, filed Apr. 11, 2006, and 60/798,103, filed May 4, 2006, the disclosures of which are incorporated herein in their entireties.

All publications and other references mentioned herein are incorporated by reference in their entireties.

Those skilled in the art will recognize, or be able to ascertain using no more than routine experimentation, numerous equivalents to the specific method and reagents described herein. Such equivalents are considered to be within the scope of this invention and are covered by the following claims.

What is claimed is:

1. A method for identifying a ligand that binds a target progenitor cell, comprising the steps of:
(i) providing a ligand display library comprising a plurality of display packages, each display package comprising at least one test ligand disposed on the surface of the display package;
(ii) contacting the display library with the target progenitor cell;
(iii) allowing the progenitor cell to differentiate; and
(iv) identifying the at least one test ligand disposed on the surface of a display package associated with a differentiated cell.

2. The method of claim 1, further comprising the step of isolating a differentiated cell with an associated display package prior to identifying the at least one test ligand.

3. The method of claim 1, wherein the associated display package is bound to the surface of the differentiated cell.

4. The method of claim 1, wherein the associated display package has been internalized into the differentiated cell by receptor-mediated endocytosis.

5-16. (canceled)

17. The method of claim 1, wherein the display package comprises no more than 5-10%, no more than 2%, or no more than 1% polyvalent displays.

18. The method of claim 1, wherein the at least one test ligand disposed on the surface of the display package is a peptide ligand.

19. (canceled)

20. The method of claim 1, wherein the plurality of display packages is a plurality of phage particles.

21-24. (canceled)

25. The method of claim 1, wherein the ligand display library comprises at least 10, at least 100, at least 1000, or at least 10,000 different display packages, each display package comprising at least one test ligand disposed on the surface of the display package.

26. The method of claim 1, wherein the display package associated with the differentiated cell is identified at least 1 day, at least 2 days, at least 4 days, at least 6 days, at least 12 days, or at least 18 days after contacting the display packages with the target progenitor cell.

27-30. (canceled)

31. The method of claim 1, wherein the target progenitor cell is a human embryo-derived cell.

32-40. (canceled)

41. A ligand identified by the method of claim 1.

42. A target progenitor cell that selectively binds the ligand of claim 41.

43. A method for identifying a target progenitor cell, comprising the steps of:
(i) providing a ligand display library comprising a plurality of display packages, each display package comprising at least one test ligand disposed on the surface of the display package;
(ii) contacting the display library with a target progenitor cell;
(iii) allowing the progenitor cell to differentiate; and
(iv) identifying a differentiated cell that associates a display package.

44. The method of claim 43, further comprising the step of identifying the at least one test ligand disposed on the surface of the associated display package.

45. The method of claim 43, wherein the associated display package is bound to the surface of the differentiated cell.

46. The method of claim 43, wherein the associated display package has been internalized into the differentiated cell by receptor-mediated endocytosis.

47-58. (canceled)

59. The method of claim 43, wherein the display package comprises no more than 5-10%, no more than 2%, or no more than 1% polyvalent displays.

60. The method of claim 43, wherein the at least one test ligand disposed on the surface of the display package is a peptide ligand.

61. (canceled)

62. The method of claim 43, wherein the plurality of display packages is a plurality of phage particles.

63-66. (canceled)

67. The method of claim 43, wherein the ligand display library comprises at least 10, at least 100, at least 1000, or at least 10,000 different display packages, each display package comprising at least one test ligand disposed on the surface of the display package.

68. The method of claim 43, wherein the differentiated cell is identified at least 1 day, at least 2 days, at least 4 days, at least 6 days, at least 12 days, or at least 18 days after contacting the display packages with the target progenitor cell.

69-72. (canceled)

73. The method of claim 43, wherein the target progenitor cell is a human embryo-derived cell.

74-82. (canceled)

83. A cell identified by the method of claim 43.
